(19) United States
(12) Patent Application Publication
(10) Pub. No.: US 2024/0306929 A1
(43) Pub. Date: Sep. 19, 2024

(54) METHOD AND SYSTEM FOR DETERMINING WHETHER A BALLOON CATHETER IS POSITIONED IN THE STOMACH OF A PERSON

(71) Applicant: Vipun Medical NV, Mechelen (BE)

(72) Inventors: Pieter Janssen, Boortmeerbeek (BE); Nico Van Tichelen, Aartselaar (BE); Carl Van Lierde, Meerbeke (BE)

(73) Assignee: Vipun Medical NV, Mechelen (BE)

(21) Appl. No.: 18/272,282

(22) PCT Filed: Jan. 18, 2022
(86) PCT No.: PCT/EP2022/051006

§ 371 Date: Jul. 13, 2023

(30) Foreign Application Priority Data
Jan. 18, 2021 (EP) 21152128.1

(51) Int. Cl. A61B 5/06; A61M 25/10

(52) U.S. Cl. CPC A61B 5/061; A61M 25/10184; A61M 2210/1053

(57) ABSTRACT

Method of determining whether a balloon of a balloon catheter is positioned in the stomach of a person. The balloon catheter comprises a catheter and a balloon. The method comprises the steps of: a) inflating (1401) the balloon and determining (1402) a pressure inside the balloon, until at least one of the following conditions is satisfied: (i) a predefined target volume is inserted in the balloon, and (ii) the determined pressure is larger than a threshold: b) testing (1403) whether the determined pressure (p) is or was larger than the threshold, and if so, deflating (1405) the balloon; and if not, providing (1406) a signal indicative of correct positioning of the balloon catheter. A system comprising a balloon catheter (130), and an inflation device (113), and a pressure sensor, and a control unit (110) for performing such a method, and optionally a food pump (112).

### FIELD OF THE INVENTION

The present invention relates in general to the field of methods and systems for determining or testing or evaluating or verifying or establishing or assessing whether a balloon catheter is positioned in the stomach of a person.

### BACKGROUND OF THE INVENTION

The stomach is a central organ in the gastrointestinal system and a major player in the food processing chain. Impaired motility and emptying are important pathophysiological factors involved in the intolerance of enteral feeding in critically ill patients but also in different gastrointestinal diseases and disorders such as gastroparesis and functional dyspepsia.

Tubes for enteral feeding, more in particular for providing nutrients into the stomach, or directly into the duodenum are known in the art. Such tubes may be entered via the nose of a patient, or via the mouth, and are typically connected to a feeding pump.

Providing nutrients directly in the stomach of patients via enteral feeding tubes might cause complications if the stomach does not function as normal. It is therefore important to ensure that the patient's gastrointestinal system is functioning.

Methods and Systems for determining whether a balloon of a balloon catheter is correctly positioned in the stomach are known in the art.

WO2008121603 mentions (in its background-section) the use of X-ray scan to confirm the position of a balloon catheter.

U.S. Pat. No. 5,431,640A describes the use of a magnet for exerting a traction force, and Litmus paper and pH measurement to determine the position in the stomach, but also in this document, final confirmation is performed by X-ray (col 8 Line 64).

EP2192885B1 describes the use of a fiberscope to eliminate the need for X-rays to be taken to verify the location of the catheter. This document also mentions a pH sensor, and an electromagnet to monitor the presence of a magnetic tip.

Several review papers discuss the issue of correct feeding tube placement in the stomach and the techniques currently available to do so, for example Taylor et al 2021 “X-ray checks of NG tube position: a case for guided tube placement”; Sanaie et al 2017 “Nasogastric tube insertion in anaesthetized patients: a comprehensive review”; and Gerritsen et al 2015 “Systematic review on bedside electromagnetic-guided, endoscopic, and fluoroscopic placement of naso-enteral feeding tubes” provide reviews on the issue.

In preferred embodiments of the present invention, the balloon of the balloon catheter used in the present invention is the same or similar to the balloon described in the following documents:

WO2019030312(A1) describes a balloon catheter which can be inserted via the nose. The balloon catheter comprises a catheter and one or more balloons fixedly attached to the catheter. As described therein, the balloon(s) may have an outer diameter from 4 to 7 cm, and may be made of a relatively hard material (e.g. durometer 70 to 100 shore A), and may have an inner volume from 90 to 330 ml when inflated by 0.2 psi (about 1379 Pa). This publication also describes a system comprising the balloon catheter, and a pressure sensor for measuring a pressure of a fluid inside the balloon, and optionally a fluid pump for selectively inflating and deflating the balloon, and a control unit for reading the pressure sensor and optionally for controlling the fluid pump.

WO2019219700(A1) describes a system for determining gastric motility and for feeding a patient, using such a balloon catheter.

It is a challenge, however, to correctly position feeding tubes.

Although insertion of feeding tubes is standard practice, a 2% incidence of trachea-pulmonary complications from feeding tube insertion has been reported (Rassias, Crit care 1998; 2(1): 25-28). While the incidence of mispositioned feeding tube may be limited, the consequences are very severe, as it can lead to fatal pulmonary complications such as pneumothorax, pleural effusion, retropharyngeal and lung abscess. It is therefore of utmost importance to correctly insert the feeding tube, and to be able to determine with (ideally) absolute certainty or (in practice) a high or very high degree of probability that the feeding tube is correctly positioned before starting enteral feeding. While several technical solutions have been described that may help the health care provider to correctly position the feeding tube, e.g. by using camera-guided placement, magnetically guided placement, or by means of impedance measurements, as far as is known to the inventors, none of these techniques provides absolute certainty or a sufficient degree of certainty. Abdominal radiography (i.e. an X-ray scan) is considered the “gold standard” for determining the position of a nasogastric tube, especially in a critically ill, elderly, dysphagic or unconscious patient. Nevertheless, such radiographic assessment is also known to yield false negatives. Moreover it implies subjecting patients to radiation, which in itself is undesirable.

In addition to being able to determine that a feeding catheter is correctly positioned directly upon initial placement, it is equally important to guarantee that this correct position is maintained during the entire period of treatment. Indeed, it happens that feeding tubes end up in an undesirable position when patients or health care professionals inadvertently pull the tube. The tube may then end up in the oesophagus, throat or outside of the body. This may occur for example when sedated patients awaken and are agitated, or while nurses are washing the patient, or during medical examination (such as X-rays or MRIs), especially when patients have to be repositioned. For this reason, X-ray scans are typically performed on a frequent basis to verify again and again that the feeding tube is still correctly positioned.

There is always room for alternatives or improvements.

### SUMMARY OF THE INVENTION

It is an object of embodiments of the present invention to provide a method and a system for determining or testing or evaluating or verifying or confirming or establishing whether a balloon of a balloon catheter is positioned in the stomach of a person.

It is an object of embodiments of the present invention to provide a method and a system for determining or testing or evaluating or verifying or confirming or establishing whether a balloon made of a relatively hard material (e.g. durometer 70 to 100 shore A), and having an outer diameter from 1.0 to 7.0 cm, and having a nominal inner volume from 0.5 to 330 ml when inflated by 0.2 psi (approximately 1379 Pa), as part of a balloon catheter, is positioned in the stomach of a person.

It is an object of embodiments of the present invention to provide a method and a system for determining or testing or evaluating or verifying or confirming or establishing whether a balloon of a balloon catheter is located or situated or positioned in a stomach of a person, while inflating the balloon to its nominal volume or target volume (a definition of “nominal volume” or “target volume” is given further).

It is also an object of embodiments of the present invention, to provide a system that is furthermore capable of providing enteral feeding and/or delivering medication to the person.

It is also an object of embodiments of the present invention to provide a computer program product for performing such a method (i.e. for verifying or determining or confirming or establishing whether the balloon of a balloon catheter is positioned in a stomach of a person).

It is an object of particular embodiments of the present invention to provide a method and a system for testing whether the balloon of the balloon catheter is positioned in the stomach of a person, and for providing a result within 5 minutes.

These and other objectives are accomplished by a method, and a system, and a computer program product according to embodiments of the present invention.

According to a first aspect, the present invention provides a method of determining or testing or evaluating or verifying or establishing or assessing whether a balloon of a balloon catheter is positioned in a stomach of a (living) person, the method comprising the steps of: a) inserting an amount of fluid in the balloon; b) determining a pressure (e.g. a momentary pressure, or a minimum pressure, or a baseline pressure) inside the balloon; c) testing if the determined pressure is larger than a threshold (e.g. p40; p30), and if an outcome of this test is true, e) deflating the balloon; otherwise continuing with step d); d) testing if a predefined target volume is injected in the balloon, and if an outcome of this test is true (meaning that the predefined target volume is injected in the balloon), outputting a signal indicating that the balloon is situated in the stomach; otherwise (meaning that the predefined target volume is not yet inserted in the balloon) going back to step a), and continuing until the pressure is larger than the threshold in step c), or until the target volume is inserted into the balloon in step e).

The “threshold” may be a predefined constant, or may be a predefined value dependent on data from the patient (e.g. sex, length, weight), or may be dynamically adjusted dependent on the amount of fluid already inserted or injected into the balloon (e.g. extracted from a one-dimensional array of values or a two-dimensional array of values).

This method has two possible outcomes: (i) in case the balloon is inflated to its target volume without the pressure being larger than the threshold, then it is established with a high degree of reliability (e.g. higher than 95%) that the balloon catheter is positioned in the stomach, (ii) in case the pressure was found to be larger than the threshold, then the balloon catheter may or may not be positioned in the stomach, and the algorithm may output a message to indicate that the position is not confirmed, but is undecided. Case (ii) will for example occur if the balloon catheter is placed e.g. in the esophagus, or in the trachea, or in the lungs, but may also occur in case the balloon catheter is placed in the stomach, but the person has coughed, or in the event of stomach contractions, or the like.

It is an advantage of this method that, in case the algorithm succeeds in fully inflating the balloon to its target volume without the pressure being larger than the threshold, it is established with a high degree of reliability (or with a high confidence level) that the balloon catheter is correctly positioned.

It is a major advantage that this method can be performed relatively fast (typically within a few minutes), and can be performed using relatively simple equipment (in contrast to for example X-ray equipment), and does not require the patient to be transported to a special room (time savings for medical personnel), and does not expose the patient to radiation.

It is an advantage that the balloon is automatic deflated in step d), which reduces the discomfort or inconvenience or risk of injury for the patient.

After deflation, meaning that the balloon catheter may not be positioned in the stomach, the medical personnel may decide to retry to inflate the balloon without repositioning the catheter, or may decide to retry after repositioning the catheter.

It is noted that in the formulation of claim 1, the notion of “time” is not explicitly mentioned. Indeed, step a) can mean for example inflating the balloon in a continuous manner, or in a discrete number of steps.

The balloon catheter may comprise a catheter and an inflatable balloon fixedly attached to said catheter. The balloon may be made of a material that shows little or no intrinsic pressure until the balloon is inflated to its target volume. The balloon may be made of a material having a durometer in the range from 70 to 100 shore A. The balloon may have an overall spherical shape with a diameter in the range from 1.0 cm to 7.0 cm (i.e. having a volume from about 0.5 ml to about 178 ml), or may have an overall spherical shape with a diameter in the range from 2.0 cm to 7.0 cm (i.e. having a volume from about 4 ml to about 178 ml), or may have an overall spherical shape with a diameter in the range from 3.0 cm to 7.0 cm (i.e. having a volume from about 14 ml to about 178 ml), or may have an overall spherical shape with a diameter from 3.5 cm to 7.0 cm (i.e. having a volume from about 22 ml to about 178 ml), or may have an overall spherical shape with a diameter from 4.0 cm to 7.0 cm (i.e. having a volume from about 33 ml to about 178 ml), or may have a non-spherical shape with a cylindrical portion having a diameter in the range from 2.0 cm to 7.0 cm and an overall volume in the range from about 4 ml to about 330 ml, or may have a non-spherical shape with a cylindrical portion having a diameter in the range from 3.0 to 7.0 cm and an overall volume in the range from about 90 ml to about 330 ml, e.g. from about 160 to about 235 ml, or may have an oblong or substantially ellipsoid shape with a diameter from about 1.0 to about 7.0 cm and an overall volume in the range from about 1.0 to about 330 ml, when inflated by a pressure of 0.20 psi (or about 10.34 mmHg, or about 1379 Pa) in an environment of 20° C. and 1013 mbar absent a counter-pressure.

The threshold may be a single, predetermined value.

The balloon catheter may further comprise at least one lumen which is fluidly connected to the inside of the balloon for selectively inflating and deflating the balloon. The balloon catheter may further comprise a second lumen which is fluidly connected to one or more feeding opening situated outside of the balloon, at a distal end of the catheter. This lumen can be used for enteral feeding.

In an embodiment, step e) further comprises: showing a graphical representation of the pressure (e.g. a graph showing pressure versus volume, or pressure versus time), and/or outputting a value of the pressure that caused the procedure to be aborted, and/or an indication (e.g. a percentage) of how far the balloon was inflated when the procedure was aborted.

While not required for allowing the method to determine with a high degree of reliability that the balloon catheter is positioned in the stomach, such indication may be useful for medical personnel when deciding whether or not to repositioning the catheter before retrying.

In an embodiment, step a) further comprises: determining the threshold (e.g. p40 or p30) to be used in step c) as a function of the amount of fluid inserted in the balloon, or as a function of time, or as a function of an iteration number.

In an embodiment, the threshold assumes at least two different values for an inserted volume from 0% to 100% of the nominal volume. For example: a first threshold value for an inserted volume from 0% to 50% of the nominal volume, and a second threshold value different from the first threshold value, for an inserted volume from 50% to 100% of the nominal volume.

In an embodiment, step b) comprises: measuring a momentary pressure inside the balloon, and considering the momentary pressure as the determined pressure; and wherein step c) comprises: testing if the momentary pressure is larger than a first threshold (e.g. “p40”).

It is an advantage of this method that the momentary pressure is compared to a threshold value, and that no “baseline pressure” or some “time averaged pressure” or a “filtered version of the pressure” needs to be calculated. Thus this algorithm can be kept extremely simple.

Another advantage of this method is that it allows to inflate the balloon relatively fast (e.g. between two stomach contractions), in which case the time required for determining that the balloon catheter is correctly positioned is also relatively fast.

It is an advantage of this method that, as soon as it is detected that the pressure is larger than said threshold, the balloon can be deflated without further delay. In practice, there will always be a small delay to perform the measurement and the comparison, but this delay is preferably smaller than 10 s, or smaller than 5 s, or smaller than 4 s, or smaller than 3 s, or smaller than 2.0 s, or smaller than 1.0 s.

A disadvantage of this method is that, in the event of one or more stomach contractions, chances are that the momentary pressure will (at some point in time) be larger than the threshold before the balloon can be fully inflated to its target volume, resulting in an outcome which is undecided.

In an embodiment, step b) comprises: measuring a plurality of momentary values of the pressure inside the balloon, and calculating a filtered pressure value based on said plurality of values; and step c) comprises: testing if the filtered pressure value is larger than a second threshold (e.g. “p30”).

In this embodiment, the pressure which is compared with the threshold is not a momentary pressure value, but a filtered pressure value. Many forms of filtering are envisioned, some of which are described further, e.g. “a low-pass filtered value”, or “a moving average”, or “a minimum value during a time interval” referred to herein as “moving minimum”, but the present invention is not limited to these forms of filtering, and other forms may also be used, for example band-pass filtering (e.g. to reduce a breathing signal), or a lower envelope as shown in FIG. 18 of WO2019219700(A1).

In an embodiment, step b) comprises: measuring a plurality of pressure values during a predefined time period or time window, and calculating an minimum pressure, or calculating a moving minimum pressure during a predefined time-window, and considering this minimum or moving minimum as the filtered pressure value.

It is a major advantage of using a minimum-filter, because it can reduce the peak pressure of gastric contractions, thereby reducing the probability that the pressure would inadvertently increase above the threshold due to the occurrence of a gastric contraction.

In an embodiment, step b) comprises: measuring a plurality of pressure values during a predefined time period or time window, and calculating an average pressure, or calculating a moving average pressure during a predefined time-window, and considering this average or moving average as the filtered pressure value.

It is an advantage of using an average or moving average that it can filter out or at least reduce unwanted signals, such as noise or such as a breathing signal. By filtering out such unwanted signals, the probability that the pressure would inadvertently increase above the threshold due to a short event, and hence the procedure is unnecessary aborted, is reduced.

It is an advantage of using an average or moving average rather than a minimum or moving minimum, in that it may sooner detect situations where the balloon is incorrectly positioned (i.e. not in the stomach), which may be masked by the minimum or moving minimum pressure.

In an embodiment, the “time window” has a duration in the range from 2 s to 60 s, or in the range from 5 to 50 s, or in the range from 5 to 40 s, or in the range from 5 s to 30 s, or in the range from 10 s to 30 s, e.g. equal to about 10 s, or equal to about 15 s, or equal to about 20 s, or equal to about 25 s, or equal to about 30 s, or equal to about 35 s.

It is an advantage of choosing a relatively small time window (e.g. from 2 to 10 seconds, e.g. of about 5 s), because in case of an incorrect placement of the catheter, the delay after which the balloon will be deflated is relatively short. It is an advantage of choosing a relatively large time window (e.g. larger than 20 s, or larger than 30 s), because it reduces or filters out a breathing signal, and may provide a better “baseline pressure”. The skilled person having the benefit of the present disclosure can easily find a good compromise. It is an advantage of using a time window of at least 15 s or at least 20 s, or at least 25 s, because it allows to partially or even completely filter out gastric peaks, or at least to strongly reduce their maximum value, and thus allows to calculate a “baseline pressure” as a minimum pressure excluding or between gastric contractions.

In an embodiment, step a) comprises: determining an amount of fluid to be inserted, and inserting the determined amount of fluid into the balloon; and step e) comprises: summing the determined amounts of fluid and comparing the summed amount with the predefined nominal volume, or determining the number of the iteration, and comparing this number with a predefined target number.

In an embodiment, the amount of fluid to be inserted may be the same (constant) for each iteration (e.g. in steps of 5 ml or in steps of 10 ml), or may vary for each iteration.

In an embodiment, the amount of fluid to be inserted in each iteration may vary, depending on the iteration step. The values of these amounts of fluid may be stored for example in a table or an array as part of a software program of a controller that controls the inflation device or in a volatile or non-volatile memory embedded or connected to said controller, where each iteration number corresponds to a predefined amount to be injected. In such a case, step e) may simply comprise: checking the number of the iteration, and if the “target number” or “final number” is reached, concluding that the target volume is inserted in the balloon.

In an embodiment, the amount of fluid to be inserted in each iteration is a fixed amount, independent of the number of the iteration.

In an embodiment, step a) and b) and c) are performed substantially simultaneously.

An example of this embodiment is illustrated in FIG. 15, where step q) comprises: continuously inflating a balloon while measuring a momentary pressure, until (at any moment in time) the target volume of the balloon is reached or until the measured momentary pressure is larger than the first pressure threshold (p40), which corresponds to the steps a) and b) and c) of FIG. 14.

With “substantially simultaneously” is meant for example that a pump is continuously operating, and that the momentary pressure is measured and compared to the first threshold at a rate of at least 1.0 Hz, or at least 2.0 Hz, or at least 5 Hz, or at least 10 Hz, and that the accumulated volume (or the iteration step) is compared to the first threshold at said rate.

In an embodiment, the method further comprises: filtering the measured pressure, e.g. using a band-pass filter, or using a sliding-minimum filter over a predefined time-window, or using a sliding-average filter over a predefined time-window, and comparing the filtered pressure with said threshold.

In an embodiment, step a) is performed during first time periods; and step b) is performed during second time periods; and the first and second time periods are at least partially (e.g. only partially, or completely) overlapping, or are mainly overlapping, or coincide.

In an embodiment, step a) is performed during first time periods; and step b) is performed during second time periods; and the first and second time periods are non-overlapping.

In this embodiment inserting fluid into the balloon (in step a) and measuring pressure (step b) are performed alternatingly, at different moments in time. This offers the advantage that any overpressure for inflating the balloon is absent, and the measured pressure may be more accurate or more stable.

According to a second aspect, the present invention also provides a method of determining (or testing or evaluating or verifying or establishing or assessing) whether a balloon of a balloon catheter is positioned in a stomach of a (living) person, comprising the steps of: i) performing the method described above as the “fast method” as a first attempt to inflate the balloon to its nominal volume without the momentary pressure becoming larger than the first threshold (e.g. p40); ii) in case the balloon was deflated in step e) of the first attempt, performing one of the methods described above as “the slow method” as a second attempt to inflate the balloon to its nominal volume without the filtered pressure becoming larger than the second threshold (e.g. p30).

It is an advantage of this method that first an attempt is made to inflate the balloon using “the fast procedure” (see e.g. FIG. 15) because it is faster, and which will likely succeed in a majority of the cases (estimated more than 80%) to confirm that the balloon catheter is positioned in the stomach, and that, in case the “fast procedure” fails, a second procedure is followed, which requires more time, but has a much higher chance of being able to fully inflate the balloon, and thereby establishing that the balloon is positioned in the stomach, even in the presence of gastric contractions.

According to a third aspect, the present invention also provides a system for determining (or testing or evaluating or verifying or establishing or assessing) whether a balloon of a balloon catheter is positioned in a stomach of a (living) person, the system comprising: the balloon catheter comprising a catheter and an inflatable balloon fixedly attached to said catheter; an inflation device (e.g. an air pump of a syringe) operatively connected to the balloon; a pressure sensor operatively connected to the balloon and configured for measuring a pressure inside the balloon; an output device for providing a signal (e.g. a visible signal or an audible signal) to the operator; a control unit connected to the pressure sensor for reading a pressure measured by the pressure sensor, and operatively connected to the inflation device for selectively inflating or deflating the balloon, and connected to the output device for providing a signal for indicating that the balloon is positioned in the stomach; wherein the control unit is configured for performing a method according to the first or second aspect, meaning that: the control unit is configured for performing an algorithm comprising the following steps: a) inserting an amount of fluid in the balloon; b) determining a pressure inside the balloon; c) testing if the determined pressure is larger than a threshold, and if an outcome of this test is true, e) deflating the balloon; otherwise continuing with step d); d) testing if a predefined nominal volume is injected in the balloon, and if an outcome of this test is true, outputting a signal indicating that the balloon is situated in the stomach; otherwise going back to step a), and continuing until the pressure is larger than the threshold (e.g. p40; p30) in step c), or until the nominal volume is inserted into the balloon in step e).

An example of such a system is shown in FIG. 1, albeit that the system shown in FIG. 1 has further components or elements (e.g. a food pump) which are not absolutely required for assessing whether the balloon catheter is positioned in the stomach.

The balloon may be made of a material that shows little or no intrinsic pressure until inflated to its target volume.

The balloon may be made of a material having a durometer in the range from 70 to 100 shore A, and being adapted to have an outer diameter in the range from 1.0 to 7.0 cm when inflated to its nominal volume.

The balloon catheter may be suitable for delivery of said inflatable balloon to a stomach of a person via the nose of said person. In this case the external diameter of the balloon catheter, when said balloon is deflated, is preferably such that the balloon catheter can pass through a hole having a diameter of about 7.7 mm (23 French). This is however not absolutely required, and the invention will also work for a balloon catheter which is inserted via the mouth of the person.

The catheter contains at least a first lumen which is fluidly coupled to the inside of the balloon via at least one first opening in the surface of the catheter. The catheter preferably further contains also a second lumen for providing food to the patient, but this is not absolutely required for the present invention to work.

The balloon may be adapted for having an outer diameter in the range from 1.0 cm to 7.0 cm when inflated to its nominal volume, or from 3.0 cm to 5.0 cm, for example equal to about 1.0 cm, or equal to about 1.5 cm, or equal to about 2.0 cm, or equal to about 2.5 cm, or equal to about 3.0 cm, or equal to about 3.5 cm, or equal to about 4.0 cm, or equal to about 4.25 cm, or equal to about 4.5 cm, or equal to about 4.75 cm, or equal to about 5.0 cm.

The balloon may be adapted for having an overall spherical shape.

The balloon may be adapted for having an effective length from 7.0 cm to 18.0 cm, or from 9.0 cm to 16.0 cm, or from 11.0 cm to 14.0 cm, or from 7.0 cm to 12.0 cm, or from 9.0 cm to 12.0 cm, or from 7.0 to 14.0 cm, or from 9.0 to 14.0 cm, or from 10.0 to 13.0 cm, when inflated by a pressure of 0.20 psi (or 1.379 kPa) in an environment of 20° C. and 1013 mbar absent a counter-pressure.

The balloon may be made of a polyurethane material having a durometer in the range from 70 to 100 shore A, or a plastic material having a durometer in the range from 25 to 100 shore D, or a plastic material having a durometer in the range from 50 to 120 rockwell R.

The balloon may be adapted to have a nominal volume in the range from 0.5 ml to 330 ml, or from 0.5 ml to 80 ml, or from 1.0 ml to 80 ml, or from 2.0 ml to 80 ml, or from 5 ml to 80 ml, or from 10 ml to 80 ml, or from 20 ml to 80 ml, or from 30 ml to 80 ml, or from 40 ml to 80 ml, or from 50 ml to 80 ml.

The balloon may be adapted to have a nominal volume in the range from 90 ml to 330 ml, or from 110 ml to 330 ml, or from 135 ml to 330 ml, or from 160 ml to 290 ml, or from 160 ml to 235 ml, or from 160 ml to 210 ml, or from 170 ml to 190 ml, for example, equal to about 150 ml, or equal to about 160 ml, or equal to about 170 ml, or equal to about 180 ml, or equal to about 190 ml, or equal to about 200 ml, or equal to about 210 ml.

The threshold may be a single, predetermined value.

In an embodiment, the threshold assumes at least two different values for an inserted volume from 0% to 100% of the nominal volume. For example: a first threshold value for an inserted volume from 0% to 50% of the nominal volume, and a second threshold value different from the first threshold value, for an inserted volume from 50% to 100% of the nominal volume.

In an embodiment, step a) further comprises: determining the threshold (e.g. p40; p30) to be used in step c) as a function of the amount of fluid inserted in the balloon, or as a function of time, or as a function of an iteration number. In this embodiment, the threshold is dynamically determined.

In an embodiment, step b) comprises: measuring a momentary pressure (e.g. pmom) inside the balloon, and considering the momentary pressure as the determined pressure; and wherein step c) comprises: testing if the momentary pressure (e.g. pmom) is larger than a first threshold (e.g. p40).

In an embodiment, step b) comprises measuring a plurality of momentary values of the pressure inside the balloon, and calculating a filtered pressure value based on said plurality of values; and step c) comprises testing if the filtered pressure value is larger than a second threshold (e.g. p30).

In an embodiment, step b) comprises: measuring a plurality of pressure values during a predefined time period or time window, and calculating an minimum pressure, or calculating a moving minimum pressure during a predefined time-window, and considering this minimum or moving minimum as the filtered pressure value.

In an embodiment, step b) comprises: measuring a plurality of pressure values during a predefined time period or time window, and calculating an average pressure, or calculating a moving average pressure during a predefined time-window, and considering this average or moving average as the filtered pressure value.

In an embodiment, step a) comprises: determining an amount of fluid to be inserted, and inserting the determined amount of fluid into the balloon; and step e) comprises: summing the determined amounts of fluid and comparing the summed amount with the predefined nominal volume, or determining a number of the iteration, and comparing this number with a predefined target number.

In an embodiment, the control unit is configured for performing step a) and b) and c) substantially simultaneously.

In an embodiment, the control unit is configured for performing step a) during first time periods; and the control unit is configured for performing step b) during second time periods; and the first and second time periods are at least partially overlapping.

In an embodiment, the control unit is configured for performing step a) during first time periods; and the control unit is configured for performing step b) during second time periods; and the first and second time periods are non-overlapping.

In an embodiment, the control unit is configured for inflating the balloon to its nominal volume without the momentary pressure exceeding the first threshold (e.g. p40) in a first attempt using the “fast method” described above, and in case the balloon was deflated in step e) of the first attempt, for inflating the balloon using the “slow method” described above in a second attempt to inflate the balloon to its nominal volume without the filtered pressure exceeding the second threshold (e.g. p30).

In an embodiment, the output device comprises at least one of: a speaker, a display, an LCD display, a light emitting device, one or more light emitting diodes (LEDs), a vibration element, a buzzer, a touchscreen.

In an embodiment, the system further comprises at least one of the following features: i) an input device for receiving a command from an operator; ii) a timer; iii) a digital processor.

In an embodiment, the control unit is or comprises a computer device, for example a laptop computer or a desktop computer.

In an embodiment, the control unit is or comprises a programmable microcontroller or digital signal processor (DSP).

In an embodiment, the system further comprises an input device for receiving a command from an operator. The input device may comprise for example a keyboard or a touch screen or at least one button, or a slider, or a switch. If an input device is present in the system, the system is preferably configured for performing the method upon receipt of a corresponding command from the operator. The input device is not absolutely required, however, because the system may also start after receiving power.

In an embodiment, instead of or in addition to an input device and/or an output device, the system may comprise or may further comprise a wireless transceiver (not shown), operatively connected to the control unit. The system may be configured for cooperating with a wireless device such as a smartphone, more in particular for receiving input commands from the smartphone device, and for sending output data to the smartphone device.

In an embodiment, the system further comprises a timer. This timer may be embedded in the controller, or may be situated outside the controller but connected thereto. The timer is not absolutely required for all embodiments (e.g. is not absolutely required for the method of FIG. 15).

In an embodiment, the system further comprises a valve (e.g. mechanical valve or electro-mechanical valve) configured for at least partially deflating the balloon in case the pressure inside the balloon is higher than a third predefined value.

The third predefined value may for example be a value in the range from 30 mm Hg (about 4000 Pa) to about 90 mm Hg (about 12000 Pa), for example equal to about 4000 (four thousand), 5000 (five thousand), 6000 (six thousand) Pa, for example equal to about 7000 Pa, or equal to about 8000 Pa, or equal to about 9000 Pa, or equal to about 10000 Pa, or equal to about 11000 Pa, or equal to about 12000 (twelve thousand) Pa.

In an embodiment wherein the first threshold “p40” is used, but not the second threshold “p30”, the third predefined value is preferably at least 10 mmHg (about 1333 Pa) larger than the first threshold value.

In an embodiment wherein the second threshold “p30” is used, but not the first threshold “p40”, the third predefined value is preferably at least 10 mmHg (about 1333 Pa) larger than the second threshold value.

In an embodiment wherein both the first threshold “p40” and the second threshold “p30” is used, the third predefined value is preferably at least 10 mmHg larger (about 1333 Pa) than the first threshold value.

According to a fourth aspect, the present invention also provides a computer program product comprising executable instructions, which, when being executed on a control unit of a system according to the third aspect, will perform a method according to the first or second aspect.

The computer program product may be stored on a computer readable medium, for example on a hard disk, or a CD-ROM, on a DVD, or may be stored in non-volatile memory, e.g. in a flash device, on a USB-stick, etc.

According to another aspect of the present invention, the present invention also provides a method of determining (or testing or evaluating or verifying or establishing or assessing) whether a balloon of a balloon catheter is positioned in a stomach of a (living) person, comprising the steps of: a) gradually (e.g. continuously or stepwise or incrementally or intermittently) inflating the balloon and (continuously or periodically or repeatedly) determining a pressure (e.g. a momentary pressure, a maximum, average or minimum pressure during a time window) inside the balloon until at least one of the following conditions is satisfied: (i) a predefined target volume is inserted in the balloon, and (ii) the determined pressure is larger than a threshold (e.g. p40, p30); b) testing whether the determined pressure is or was larger than the threshold (e.g. p40, p30), and if an outcome of this test is true, deflating the balloon; and if an outcome of this test is false, providing a signal indicative of correct positioning of the balloon.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiments described hereinafter.

### BRIEF DESCRIPTION OF THE DRAWINGS

FIG. 1 of the present invention is a replica of FIG. 32 of WO2019030312(A1), and shows a schematic block diagram of a system according to an embodiment of the present invention. The system shown in FIG. 1 comprises: a balloon catheter with one balloon and having two lumen: a first lumen connected to the balloon, a second lumen for feeding.

FIG. 2 of the present invention is a replica of FIG. 29 of WO2019030312(A1) with the addition of a balloon in dotted line, and shows an example of a balloon catheter comprising a balloon which is inserted via the nose, and via the esophagus into the stomach of a person.

FIG. 3 of the present invention is a replica of FIG. 21 of WO2019030312(A1), and shows a schematic representation of several material hardness scales relevant for the present invention.

FIG. 4 of the present invention is a replica of FIG. 24 of WO2019030312(A1), and shows a picture of an illustrative balloon catheter as can be used in embodiments of the present invention. The balloon shown in FIG. 4 is deflated and flattened.

FIG. 5 of the present invention is a replica of FIG. 25 of WO2019030312(A1), and shows the balloon catheter of FIG. 4, wherein the balloon is inflated to its “target volume” (also referred to herein as its “nominal volume”) at a pressure of 0.2 psi (approximately 1379 Pa).

FIG. 6 of the present invention is a replica of FIG. 26 of WO2019030312(A1), and shows a volume versus pressure profile for a balloon made of polyurethane with a durometer of 90 shore A, and having a target volume of about 150 ml, as can be used in embodiments of the present invention.

FIG. 7 of the present invention is a replica of FIG. 28 of WO2019030312(A1), and shows a volume versus pressure profile for a balloon made of polyurethane with a durometer of 90 shore A, and having a target volume of about 190 ml, as can be used in embodiments of the present invention.

FIG. 8 of the present invention is a replica of FIG. 8(a) of WO2019219700(A1), and shows an example of a raw pressure signal as can be obtained by a balloon catheter described above, e.g. using a system as shown in FIG. 1 of the present invention, or variants thereof, e.g. a variant not having the food pump.

FIG. 9 of the present invention is a replica of FIG. 9(a) of WO2019219700(A1), and shows another example of a raw pressure signal as can be obtained by a balloon catheter described above.

FIG. 10(a) shows a graph with five curves obtained from experiments using a balloon catheter as described above having a nominal volume of 150 ml, illustrating a peak value (i.e. maximum value) of the pressure measured during a certain time-window versus volume measured (white circles) when the balloon is situated on a table or bench, measured (black circles) when the balloon is situated in a stomach, and measured (black squares) when the balloon is situated in an esophagus.

FIG. 10(b) shows a curve with raw pressure measurements versus time.

FIG. 11 shows a graph with three curves obtained from experiments using a balloon catheter as shown in FIG. 5 having a nominal volume of 150 ml, showing a pressure versus volume measured when the balloon is situated inside a dummy trachea (or bench model of a trachea) having an inner diameter of about 15 mm (black circles), when the balloon is situated inside a dummy trachea having an inner diameter of about 22 mm (black squares), and when the balloon is situated inside a dummy trachea having an inner diameter of about 28 mm (black triangles). Also shown are values of Δp/ΔV (also referred to herein as “pressure derivative”).

FIG. 12 shows a graph illustrating balloon pressure versus time, obtained from a bench model of a trachea, simulating balloon pressure that would be measured in case the balloon would be situated in a trachea, when predefined volumes of air are injected in the balloon in a stepwise manner, in the absence of contractions (e.g. due to swallowing).

FIG. 13 shows a graph illustrating balloon pressure versus time, measured in the esophagus of a healthy test person, when predefined volumes of air are injected in the balloon in a stepwise manner.

FIG. 14 shows a flowchart of a general method of determining whether a balloon of a balloon catheter is positioned in a stomach of a living person, (and thus also whether the balloon catheter and/or a feeding lumen thereof, if present, is correctly positioned), as proposed by the present invention.

FIG. 15 shows a flowchart of a first specific method of determining whether a balloon of a balloon catheter is positioned in a stomach of a living person, (and thus also whether the balloon catheter and/or a feeding lumen thereof, if present, is correctly positioned), as proposed by the present invention, referred to herein as “the fast method”, which is a special case of the method of FIG. 14.

FIG. 16(a) shows an illustrative example of how the pressure inside the balloon of a balloon catheter as described above may typically vary over time, when applying the method of FIG. 15, in case the balloon is positioned in the stomach, in the absence of any gastric contractions.

FIG. 17 shows an illustrative example of how the pressure inside the balloon of a balloon catheter as described above may typically vary over time, when applying the method of FIG. 15, in case the balloon is positioned in the stomach, in the presence of gastric contractions.

FIG. 18 shows an illustrative example of how the pressure inside the balloon of a balloon catheter as described above may typically vary over time, when applying the method of FIG. 15, in case the balloon would inadvertently be situated in the trachea, irrespective of any gastric contractions.

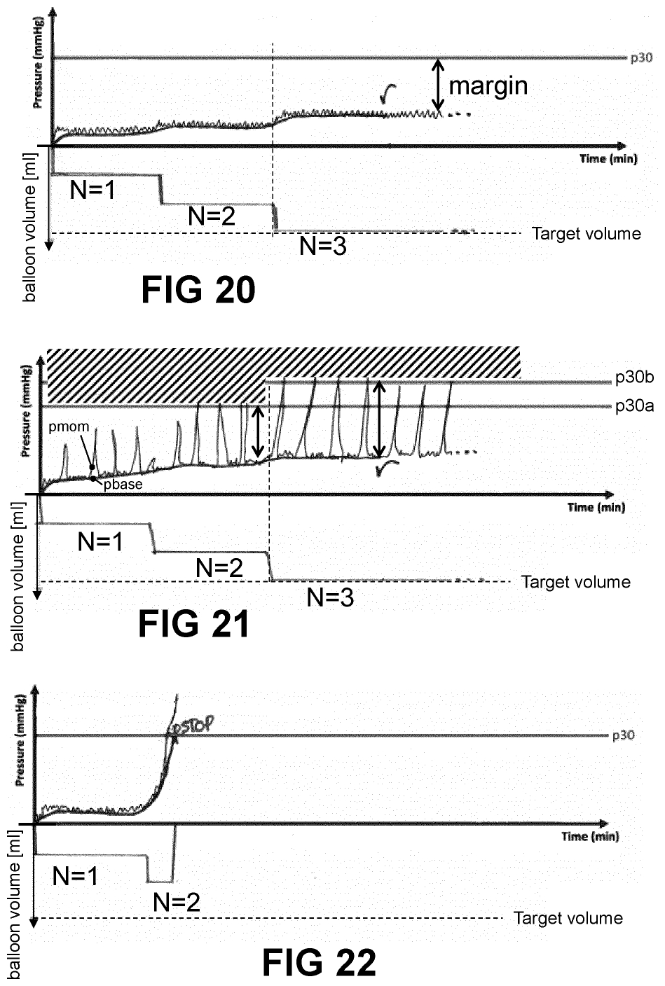

FIG. 16(b) shows a variant of FIG. 16(a) using a plurality of threshold values.

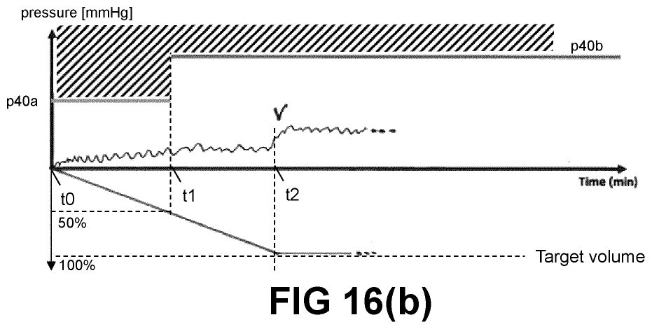

FIG. 19 shows a flowchart of a second specific method of determining whether a balloon of a balloon catheter is positioned in a stomach of a living person, as proposed by the present invention, referred to herein as “the slow method”, which is also a special case of the method of FIG. 14.

FIG. 20 shows an illustrative example of how the pressure inside the balloon of a balloon catheter as described above may typically vary over time, when applying the method of FIG. 19, in case the balloon is positioned in the stomach, in the absence of any gastric contractions.

FIG. 21 shows an illustrative example of how the pressure inside the balloon of a balloon catheter as described above may typically vary over time, when applying the method of FIG. 19, in case the balloon is positioned in the stomach, in the presence of gastric contractions.

FIG. 22 shows an illustrative example of how the pressure inside the balloon of a balloon catheter as described above may typically vary over time, when applying the method of FIG. 19, in case the balloon would inadvertently be situated in the trachea, irrespective of any gastric contractions.

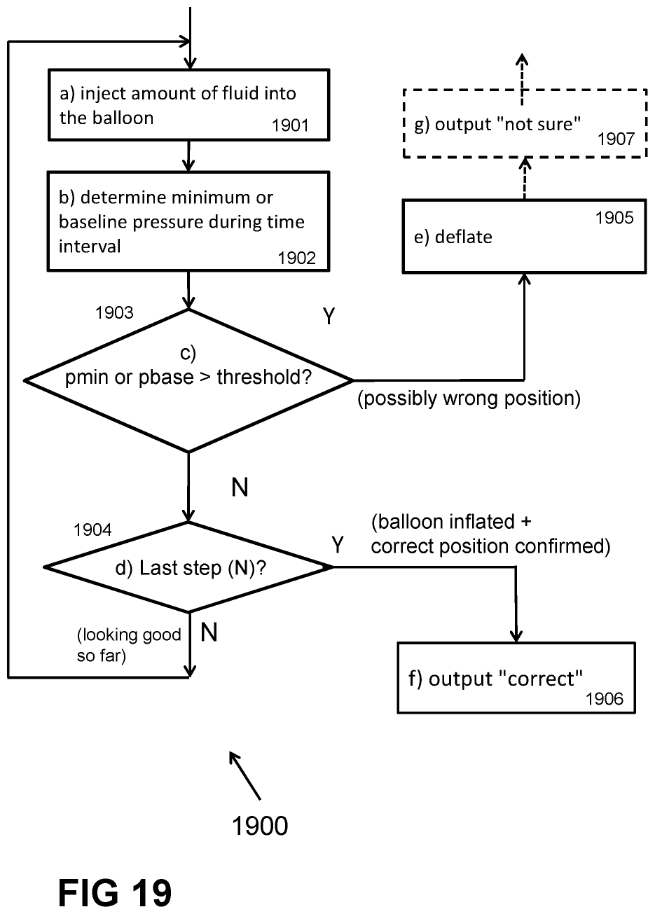

FIG. 23 shows a flowchart of a third specific method of determining whether the balloon of a balloon catheter is positioned in a stomach of a living person, as proposed by the present invention, referred to herein as “the combined method”, which is composed of the steps of the (fast) method of FIG. 15, and in case the outcome is unsuccessful, followed by the steps of the (slow) method of FIG. 19.

FIG. 24 shows an illustrative example of how the pressure inside the balloon of a balloon catheter as described above may vary over time, when applying another variant of the method of FIG. 14, in case the balloon is positioned in the stomach, in the absence of any gastric contractions.

FIG. 25 shows an illustrative example of how the pressure inside the balloon of a balloon catheter as described above may vary when applying another variant of the method of FIG. 14, in case the balloon is positioned in the stomach, in the presence of gastric contractions.

FIG. 26 shows an illustrative example of how the pressure inside the balloon of a balloon catheter as described above may vary when applying another variant of the method of FIG. 14, in case the balloon would be inadvertently situated in the trachea, irrespective of any gastric contractions.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and may not be drawn to scale for illustrative purposes. The dimensions and the relative dimensions may not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The present invention is related to methods and systems for determining or verifying or establishing or assessing whether the balloon of a balloon catheter is positioned in the stomach of a person. It is currently envisioned that the reliability of the methods proposed is at least 95%, i.e. that the likelihood that the balloon is indeed positioned in the stomach when the algorithm determines that this is the case, is correct in at least 95% of the cases.

The proposed methods and systems are particularly suitable for a balloon catheter comprising at least one, or only one inflatable balloon that shows little or no intrinsic pressure until inflated to its target volume.

In preferred embodiments, the methods and systems make use of a balloon catheter comprising at least one inflatable balloon fixedly attached to said catheter, wherein the at least one balloon is made of a material having a durometer in the range from 70 to 100 shore A. The balloon may have an outer diameter in the range from 1.0 cm to 7.0 cm when inflated to its nominal volume, e.g. an outer diameter in the range from 1.0 to 6.0 cm, or in the range from 2.0 to 6.0 cm, or in the range from 3.0 to 7.0 cm, or in the range from 3.0 to 6.0 cm, or in the range from 2.0 cm to 5.0 cm, or in the range from 3.0 cm to 5.0 cm, e.g. equal to about 1.0 cm, or equal to about 2.0 cm, or equal to about 3.0 cm, or equal to about 4.0 cm, or equal to about 5.0 cm.

The balloon catheter may further comprise a second lumen for feeding the patient, but the feeding aspect falls outside of the scope of the present invention. The interested reader may consult WO2019030312(A1) or WO2019219700(A1) for more details, both of which are included herein by reference in their entirety. A brief summary of some of the relevant aspects of these documents will be given next, as an introduction to the present invention. Before doing so, however, it is noted that these WO-documents describe balloons having an elongated shape with an outer diameter in the range from 4 cm to 7 cm, and having a nominal volume in the range from 90 ml to 330 ml, whereas the present invention also works for balloons having an outer diameter from 1.0 cm to 7.0 cm when inflated to their nominal volume, e.g. balloons having an overall spherical shape adapted to have an outer diameter in the range from 1.0 cm to 7.0 cm when inflated, and a nominal volume in the range from about 0.5 ml to about 178 ml; or balloons having an overall spherical shape adapted to have an outer diameter in the range from 3 cm to 7 cm when inflated, and having a nominal volume in the range from about 14 ml to about 178 ml; or balloons having a non-spherical shape with a cylindrical portion adapted to have an outer diameter in the range from 3 cm to 7 cm when inflated, and having a nominal volume in the range from 20 ml to 330 ml or from 70 ml to 330 ml or from 90 ml to 330 ml, or balloons having an oblong or substantially ellipsoid shape with a diameter from about 1.0 to about 7.0 cm and an overall volume in the range from about 1.0 to about 330 ml. Most other aspects mentioned in these WO-documents are also applicable here.

With “target volume” or “nominal volume” of the balloon is meant the “total inner volume” when the balloon is inflated to a pressure of 0.2 psi (about 1379 Pa) in an environment of 20° C. and 1013 mbar absent a counter-pressure.

The expression that “a balloon is (fully) inflated”, means that the balloon is inflated to its target volume or to its nominal volume, unless explicitly stated otherwise, or clear from the context that something else was meant.

In this document, pressure is expressed in psi (pounds per square inch), or in mm Hg, or in Pa.

0.2 psi is equal to about 10.34 mm Hg, and is equal to about 1379 Pa.

In this document the expression “the catheter is correctly placed” or “the balloon is correctly placed” or “the balloon is correctly placed in the stomach” means that the balloon of the balloon catheter is situated in the stomach.

In this document the expression “the balloon catheter is incorrectly placed” or “the balloon is incorrectly placed” means that the balloon of the balloon catheter is not situated in the stomach, but for example in the trachea, or the esophagus, or in the lungs.

In this document, the expression “moving minimum” means the minimum value calculated over a sliding time window having a predefined duration (in analogy with the expression “moving average”).

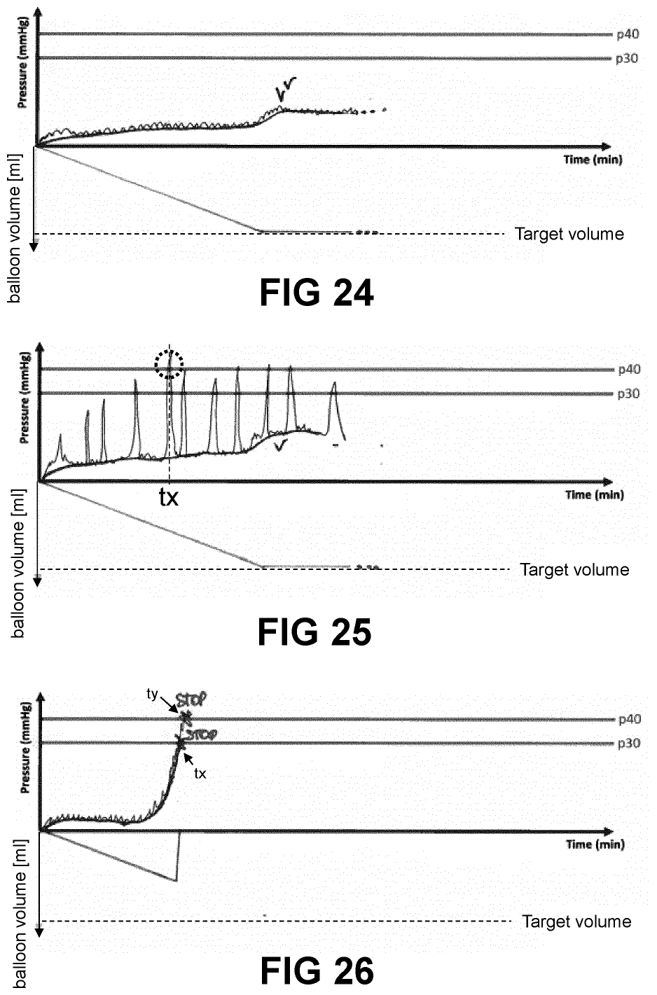

In this document the expression “baseline pressure” refers to a pressure curve connecting local minima of the original curve, or to a curve formed by “moving minima”, or to a “lower envelope” as illustrated in FIG. 18 of WO2019219700(A1), or the like.

In this document, the verbs “determining” or “testing” or “evaluating” or “verifying” or “establishing” or “assessing” or “ascertain” whether the balloon is situated in the stomach are used to indicate that the algorithm can have two possible outcomes: i) yes, ii) undecided. The algorithm is designed to output a “yes” only if the confidence level is higher than a predefined value, e.g. about 95%.

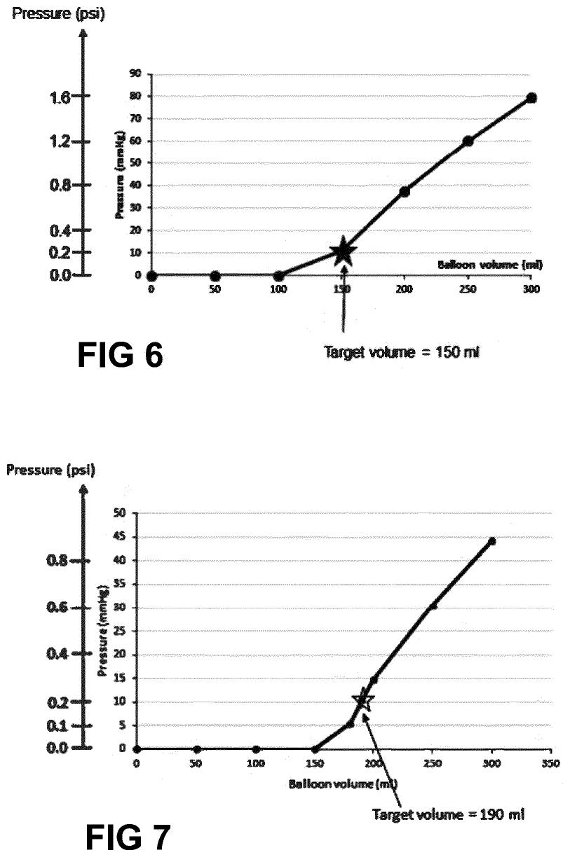

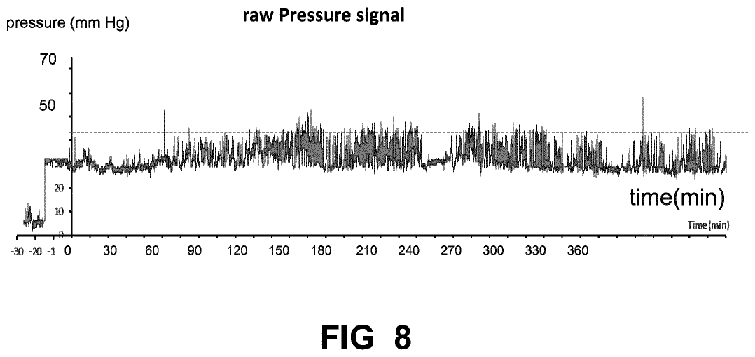

As mentioned in the background section, the balloon catheter described in WO2019030312(A1) (and illustrated in FIG. 4 and FIG. 5 of the present document) can be used to feed a patient directly in the stomach while monitoring gastric contractions. In order to monitor gastric contractility, the known balloon is inflated to its target volume (e.g. 150 ml) and to an outer diameter of minimally 4 cm to maximally 7 cm while the pressure in the balloon remains below 0.2 psi (about 1379 Pa). Gastric contractility can automatically be quantified with a system as described in WO2019219700(A1), and feeding speed can be adjusted accordingly.

For feeding catheters in general the location of the feeding opening(s) (e.g. at the distal tip) is crucial as misplacement of the catheter in the lungs for example can lead to severe consequences such as pneumothorax, pleural effusion, retropharyngeal and lung abscess. In addition to trachea-pulmonary complications, feeding tube mispositioning may occur when patients or health care professionals inadvertently pull the tube while food is being delivered to the patient. The tube may then end up in the esophagus, throat or outside the body. This may happen for example when sedated patients awaken and are agitated, or while nurses are washing the patient, or during medical examinations (such as X-ray scan or MRI-scan when the patient has to be repositioned). The correct position of a balloon catheter is even more crucial as inadvertent inflation of the balloon, for example in the lungs, might suffocate a patient. This also applies to a balloon catheter with feeding functionality, e.g. as described in WO2019030312(A1), where correct positioning of the balloon and the distal tip is crucial. X-ray scans can be performed to verify that the feeding catheter is correctly positioned upon initial placement, and thereafter on a frequent basis to verify again and again that the feeding catheter is still correctly positioned. However X-rays put a radiation burden on patients. In the prior art, several relatively complex methods have been developed to assist in correct placement of a feeding catheter, such as camera-guided placement or magnetically guided placement, or impedance measurement to determine the feeding tube position. Implementation of these techniques however would significantly complicate the clinical practice.

The inventors of the present invention wanted to find an easier way and/or a more convenient way to determine with a relatively high degree of confidence (e.g. at least 95%) whether a balloon catheter as described in WO2019030312(A1), or variants thereof (e.g. having a somewhat different shape and/or a slightly smaller nominal volume and/or a slightly smaller outer diameter) is positioned in the stomach.

They wondered whether it would be possible to determine if the balloon is positioned in the stomach on the basis of the amount of injected fluid (e.g. air) and the resulting balloon pressure, e.g. measured while gradually inflating the balloon, e.g. based solely on volume and pressure. Given the long lasting need, and given the complexity (and associated cost) of the existing solutions (X-ray scan, camera, magnets, pH measurements, . . . ), and given the severity of the injuries or complications described above, it is almost unbelievable that such a simple solution would be possible at all, let alone would provide a reliable result. But after performing several experiments, the inventors succeeded in developing several algorithms to establish whether the balloon is indeed positioned in the stomach. Moreover, the entire procedure only takes a few minutes, and does not require the patient to be exposed to X-rays.

More specifically, the present invention provides a method of determining (e.g. testing or evaluating or establishing or assessing) whether a balloon catheter is positioned in the stomach of a living person. The balloon catheter comprises a catheter, and an inflatable balloon fixedly connected to the catheter. The method comprises the steps of: a) gradually, e.g. continuously or stepwise or incrementally or intermittently inflating the balloon and continuously or periodically or repeatedly determining a pressure inside the balloon until (i) a predefined target volume is inserted in the balloon, or (ii) the determined pressure is larger than a first threshold (e.g. a first predefined threshold, or a dynamically adjustable threshold); b) testing whether the determined pressure is or was larger than the first threshold, and if an outcome of this test is false, providing a signal indicative of correct positioning of the balloon catheter.

Preferably, the method further comprises the step of automatically deflating the balloon if an outcome of the test is true. Depending on the implementation, the latter does not necessarily mean that it is established that the balloon catheter is in fact mispositioned, but rather that it may be mispositioned. Optionally, the method may provide a signal indicative of incorrect positioning, or a signal indicative of an undecided or uncertain condition, or no signal. The Health Care Provider can then retry, optionally after repositioning the balloon catheter, and optionally using another variant of the algorithm (e.g. a slow variant).

In case the outcome of the test of step b) is false, however, meaning that the pressure threshold (e.g. the predefined or dynamic pressure threshold) is not reached, even though the balloon is fully inflated to its target volume, it is guaranteed that the balloon catheter is positioned in the stomach. Several implementations of this method are possible, as will be described in more detail below.

Referring now to the figures.

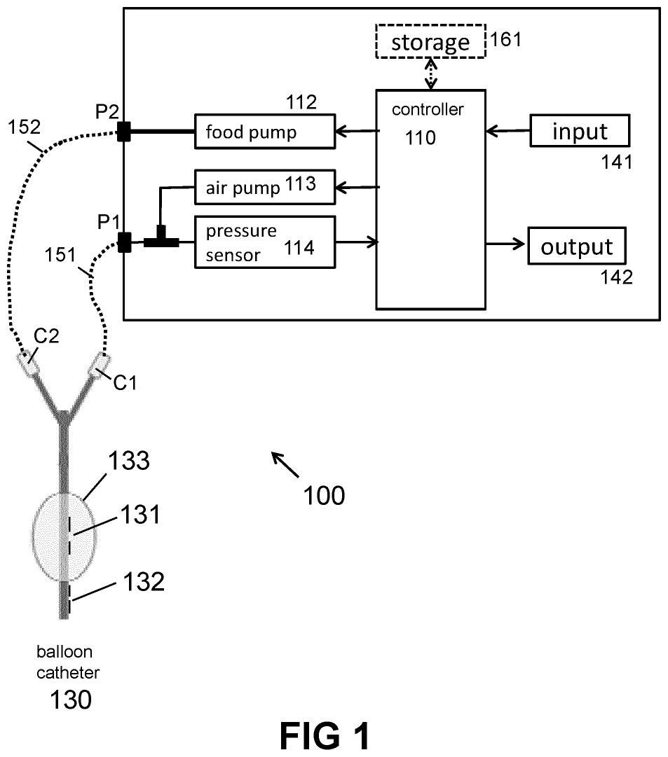

FIG. 1 of the present invention is a replica of FIG. 32 of WO2019030312(A1), and shows a schematic block diagram of a system 100 according to an embodiment of the present invention. It is noted that the system of FIG. 1 does not show the most general system proposed by the present invention, because it contains some optional components which are not absolutely required for the present invention to work.

The system 100 comprises a balloon catheter 130 comprising an inflatable balloon 133 fixedly attached to said catheter. The balloon is made of a material that shows little or no intrinsic pressure until inflated to its target volume. In preferred embodiments, the balloon 133 is made of a material having a durometer in the range from 70 to 100 shore A, and is adapted to have an outer diameter (or a maximum outer diameter) in the range from 1.0 cm to 7.0 cm when fully inflated, or a subrange hereof (as mentioned above). The system 100 of FIG. 1 further comprises an inflation device 113 (e.g. a pump or a syringe) operatively connected to the balloon 133. The system 100 further comprises a pressure sensor 114 operatively connected to the balloon 133 and configured for measuring a pressure inside the balloon. The system 100 further comprises an output device 142 for providing a signal to the operator (e.g. Health Care Provider). The system 100 further comprises a control unit 110 connected to the pressure sensor 114, and configured for obtaining a pressure signal or pressure value from the pressure sensor 114, and operatively connected to the inflation device 113 (e.g. the pump or syringe) for selectively inflating or deflating the balloon, and connected to the output device 142 for providing a signal indicative of whether the balloon catheter 130 is correctly positioned (i.e. is positioned in the stomach of the person), and optionally for providing a signal that the balloon is incorrectly positioned (i.e. is not positioned in the stomach) or that the balloon may be incorrectly positioned (if the result of the algorithm is undecided). The control unit 110 is configured for performing a method as described above, or a variant thereof, e.g. as will be described in more detail further, e.g. in FIG. 14 (general method), or FIG. 15 (fast method), or FIG. 19 (slow method), or FIG. 23 (combination method).

As shown in FIG. 1, the system 100 may further comprise a food pump 112, and the balloon catheter 130 comprises one balloon 133, and a catheter having two lumen: a first lumen 151 fluidly connected to opening 131 inside the balloon 133, and a second lumen 152 fluidly connected to one or more feeding openings 132 outside the balloon 133 for feeding. The food pump 112 and the second lumen 152 is not required for the present invention to work, i.e. to determine if the balloon catheter is positioned in the stomach, but is an optional feature, which is present in preferred embodiments.

The balloon catheter 130 of a system 100 according to embodiments of the present invention may deviate in several aspects from those described in WO2019030312(A1) and WO2019219700(A1). For example, it is not absolutely required that the balloon catheter is insertable via the nose. Indeed, the methods and systems according to the present invention will also work with a balloon catheter comprising a balloon which is inserted via the mouth. And the methods and systems proposed herein will also work for a balloon having an overall spherical shape with a diameter in the range from 1.0 cm to 7.0 cm (i.e. having a volume from about 0.5 ml to about 178 ml); or having an overall spherical shape with a diameter in the range from 2.0 cm to 7.0 cm (i.e. having a volume from about 4 ml to about 178 ml); or having an overall spherical shape with a diameter in the range from 3.0 cm to 7.0 cm (i.e. having a volume from about 14 ml to about 178 ml); or having an overall spherical shape with a diameter from 3.5 cm to 7.0 cm (i.e. having a volume from about 22 ml to about 178 ml); or having an overall spherical shape with a diameter from 4.0 cm to 7.0 cm (i.e. having a volume from about 33 ml to about 178 ml); or a balloon having a non-spherical shape with a cylindrical portion having a diameter in the range from 3.0 to 7.0 cm and an overall volume in the range from about 90 ml to about 330 ml, e.g. from about 160 to about 235 ml, when inflated by a pressure of 0.20 psi (or 1.379 kPa) in an environment of 20° C. and 1013 mbar absent a counter-pressure, and is preferably made of a material having a durometer of at least 70 shore A. In preferred embodiments, the balloon is made of a polyurethane material having a durometer in the range from 70 to 100 shore A.

The system 100 of FIG. 1 may further comprise an input device 141 for receiving commands from an operator, for example, when to start the algorithm to inflate the balloon and to determine whether the balloon is positioned in the stomach.

The system 100 of FIG. 1 further comprises a first port P1 which is connected or connectable to a first connector C1 of the balloon catheter 130 to fluidly connect the pressure sensor 114 and (if present) also the air pump 113 to the first lumen 151. The system 100 has a second port P2 which is connected or connectable to a second connector C2 of the balloon catheter 130 to fluidly connect the food pump 112 to the second lumen 152. Besides an algorithm for inflating the balloon and determining whether the balloon is positioned in the stomach as proposed by the present invention, the controller 110 may comprise further algorithms, such as for example an algorithm for calculating a motility-index, and for displaying the motility-index on the output means 142. In some embodiments, medical personnel can control the food pump 112 by providing instructions to the controller 110 via the input means 141, based on the motility information provided on the output means 142. The system may further comprise a storage device 161 for storing said pressure data and/or said motility information.

The output device 142 may comprise one or more of: a speaker, a display, an LCD display, a light emitting device, one or more light emitting diodes (LEDs), a vibration element, a buzzer, etc. The control unit may be or comprise a digital processor, or a programmable device, such as a microcontroller, or a digital signal processor (DSP), or a computer device, e.g. a laptop computer, or a desktop computer, etc. In some embodiments the controller 101 preferably has a timer (e.g. for performing the algorithm of FIG. 15 or variants thereof).

While not shown in FIG. 1, the system 100 may also comprise safety provisions, such as for example a valve, e.g. a mechanical valve or an electro-mechanical valve configured for automatically deflating or at least partially deflating the balloon 133 in case the pressure inside the balloon is higher than a predefined value, or is higher than a certain value for a predefined timer period, etc. The predefined value may be a value in the range from 45 mm Hg (about 6000 Pa) to about 90 mm Hg (about 12000 Pa), for example equal to about 6000 (six thousand) Pa, or equal to about 7000 Pa, or equal to about 8000 Pa, or equal to about 9000 Pa, or equal to about 10000 Pa, or equal to about 11000 Pa, or equal to about 12000 (twelve thousand) Pa.

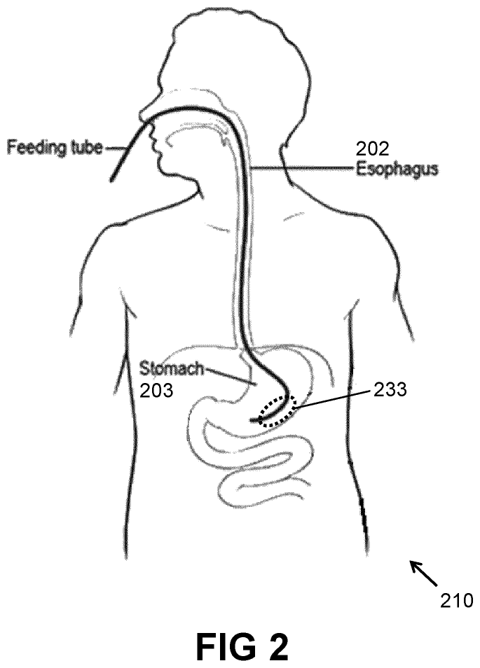

FIG. 2 of the present invention is a variant of FIG. 29 of WO2019030312(A1), and shows an example of a balloon catheter comprising a balloon 233 which is inserted via the nose, and via the esophagus 202 into the stomach 203 of a person 210. This Figure illustrates a problem underlying the present invention, namely, how can one be sure that the balloon 233 of the balloon catheter is located inside the stomach, and not in the esophagus or in the lungs of the patient. As mentioned above, this is crucial before starting to feed or provide medication to a patient. Before describing the algorithms provided by the present invention, first some characteristics of balloons as can be used in embodiments of the present invention, are described.

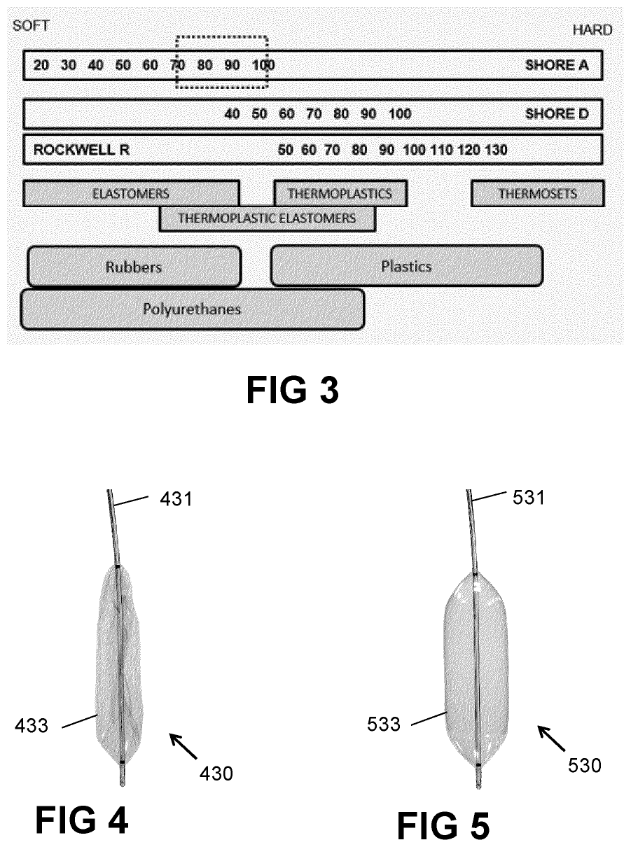

FIG. 3 of the present invention is a replica of FIG. 21 of WO2019030312(A1), and shows a schematic representation of several material hardness scales. Such scales are known per se in the art, and are provided herein only as an easy reference for the reader. It is noted that slight variations of these scales may be found in literature, but the main point of showing them here is that the scales of shore A, shore D and rockwell R overlap. Currently the most-preferred embodiments of balloons are those based on polyurethanes having a durometer of 70 to 100 shore A, as indicated by the dotted rectangle, but other materials having a similar hardness may also be used.

FIG. 4 of the present invention is a replica of FIG. 24 of WO2019030312(A1), and shows a picture of an illustrative balloon catheter 430 as can be used in embodiments of the present invention. The balloon catheter 430 comprises at least one catheter 431 and at least one balloon 433. The balloon 433 shown in FIG. 4 is deflated and flattened, and has a “target volume” (or nominal volume) of about 200 ml, and is made of a polyurethane having a durometer of 90 shore A. As can be appreciated from FIG. 4, even a material with such a relatively high hardness can be flattened and crumpled to a very small diameter, e.g. to a diameter small enough to be entered via the nose.

FIG. 5 of the present invention is a replica of FIG. 25 of WO2019030312(A1), and shows the balloon catheter 500 of FIG. 4, wherein the balloon 533 is inflated to its “target volume”, in this particular case to about 200 ml.

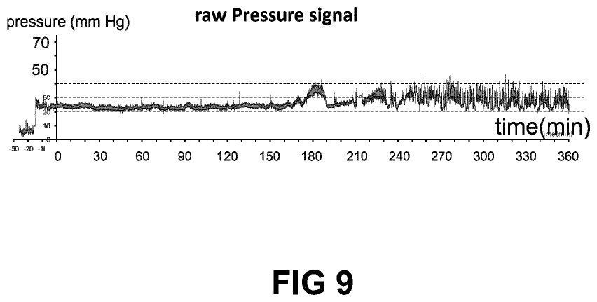

FIG. 6 of the present invention is a replica of FIG. 26 of WO2019030312(A1), and shows a volume versus pressure profile for a balloon made of polyurethane with a durometer of 90 shore A, and having a target volume of about 150 ml, as can be used in embodiments of the present invention. The balloon is inflated and deflated ‘on the bench’, outside a stomach. Hence there is no counter-pressure exerted on the balloon surface during this test. The outside of the balloon is air at room temperature (about 20° C.) and atmospheric pressure (about 1013 mbar or 101300 Pa). If the balloon is inflated with a pressure of 0.20 psi (about 1379 Pa), e.g. “on the bench”, in an environment at 20° C. and 1013 mbar, without counter-pressure exerted on the balloon surface, the volume is about 150 ml. This value is considered the target volume of this particular balloon.

As can be seen from FIG. 6, as long as the volume inserted into the balloon is lower than the target volume (in the example: 150 ml), the pressure inside the balloon is close to zero (in the absence of an external counter pressure exerted upon the balloon), and once the target volume is inserted into the balloon, the balloon pressure increases quite linearly with (additional) inserted volume. FIG. 6 illustrates a characteristic of this particular balloon made of polyurethane, but this is not the pressure that is measured during normal use, when the balloon is inside the stomach of a patient.

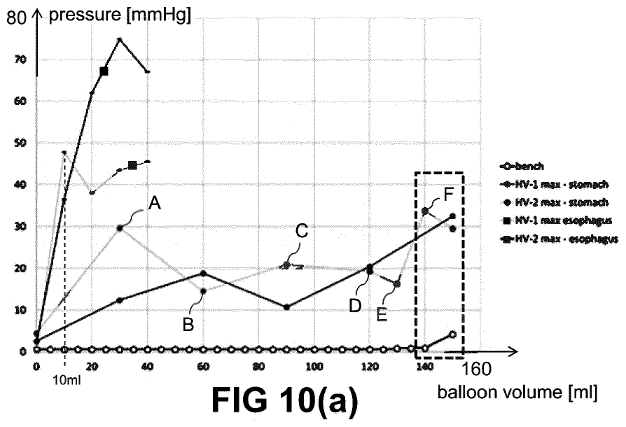

FIG. 7 of the present invention is a replica of FIG. 28 of WO2019030312(A1), and shows a volume versus pressure profile for a balloon made of polyurethane with a durometer of 90 shore A, and having a target volume of about 190 ml. Again, as long as the volume inserted into the balloon is less than the “target volume” of the balloon, (in this example: 190 ml), the pressure inside the balloon is close to zero (in the absence of a counter pressure), and once the target volume is inserted into the balloon, the balloon pressure increases quite linearly with (additional) inserted volume.

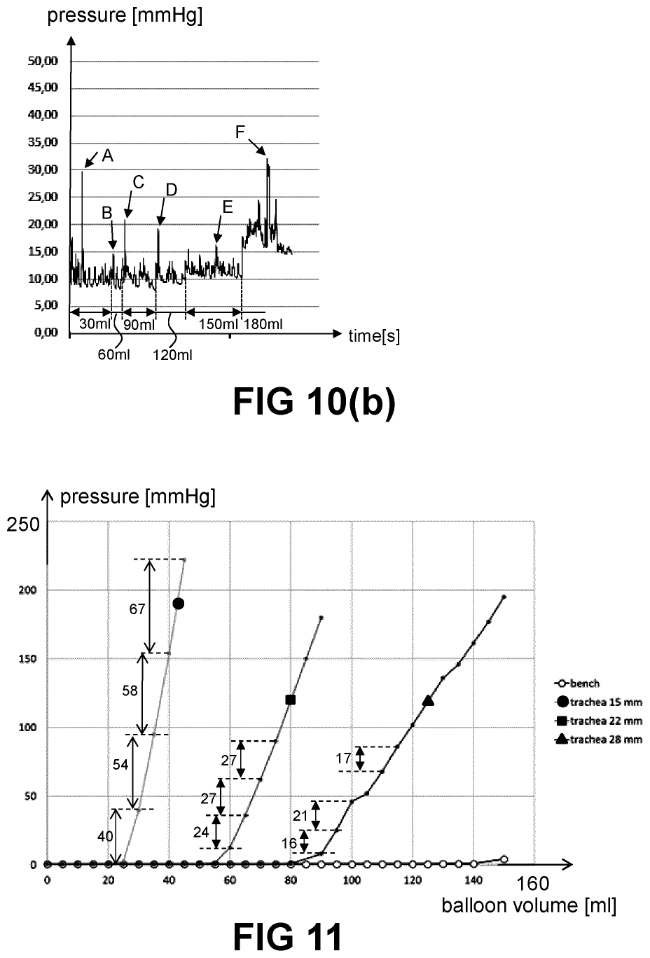

FIG. 8 of the present invention is a replica of FIG. 8(a) of WO2019219700(A1), and shows an example of a raw pressure signal as can be obtained by a balloon catheter as described above, having a balloon with a target volume of about 200 ml, using a system as shown in FIG. 1 of the present invention, or variants thereof. In the example shown, the balloon was in a partially inflated state in the period from about −25 min to −15 min, and was (rapidly) inflated to its target volume around t=−15 minutes. As can be seen, the pressure inside the balloon before inflation to its target volume is low (about 5 to 12 mmHg in this example). In the period from t=−15 minutes to t=360 minutes, the balloon is fully inflated, and it can be observed that the pressure of the inflated balloon of this particular example varies mostly in the range from about 28 mmHg to about 43 mmHg. The “minimum pressure” of this particular example is equal to about 28 mmHg, and the pressure temporarily increases with about 10 to 15 mmHg due to gastric contractions. A “baseline pressure” of this example, for example defined as the minimum value over a sliding time-window of about 1 minute, seems to be a value in the range from about 28 mm Hg to about 35 mmHg. The baseline pressure of the curve before t=−15 min is lower than about 5 mmHg.

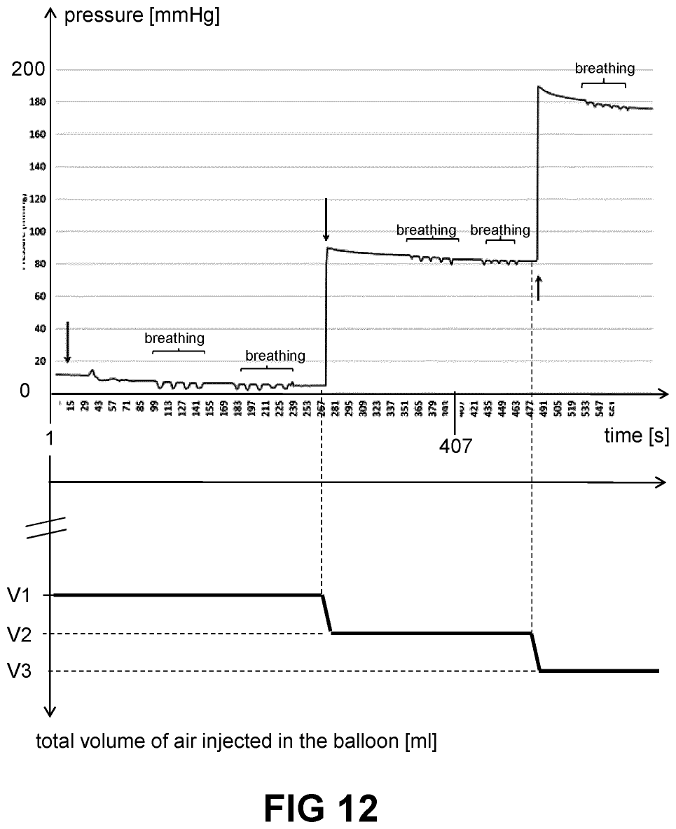

FIG. 9 of the present invention is a replica of FIG. 9(a) of WO2019219700(A1), and shows another example of a raw pressure signal as can be obtained by a balloon catheter having a balloon with a target volume of about 200 ml. Also in this example, the balloon was in a partially inflated state in the period from about −25 min to −15 min, and was (rapidly) inflated to its target volume around t=−15 minutes. As can be seen, the balloon pressure before inflation to its target volume is low (about 5 to 10 mmHg in this example). In the period from t=−15 minutes to t=360 minutes, the balloon is fully inflated, and it can be observed that the pressure inside the inflated balloon varies mostly in the range from about 20 mmHg to about 40 mmHg. The “minimum pressure” of this particular example is equal to about 20 mmHg, and the pressure temporarily increases with about 10 to 15 mmHg due to gastric contractions. A “baseline pressure” of this example, for example defined as the minimum value over a sliding time-window of about 1 minute, seems to be a value in the range from about 20 mm Hg to about 32 mm Hg. The baseline pressure of the curve before t=−15 min is lower than about 5 mmHg.

In WO2019219700(A1) a motility index is calculated based on the balloon pressure of a fully inflated balloon, i.e. based on the portions of the curves after t=−15 min of FIG. 8 and FIG. 9. The left portion of the curves before t=−15 min cannot be used to reliably measure a motility index with good accuracy. But the inventors surprisingly came to the idea to investigate whether this portion could perhaps be used for determining whether the balloon catheter is positioned in the stomach. So they started experimenting.

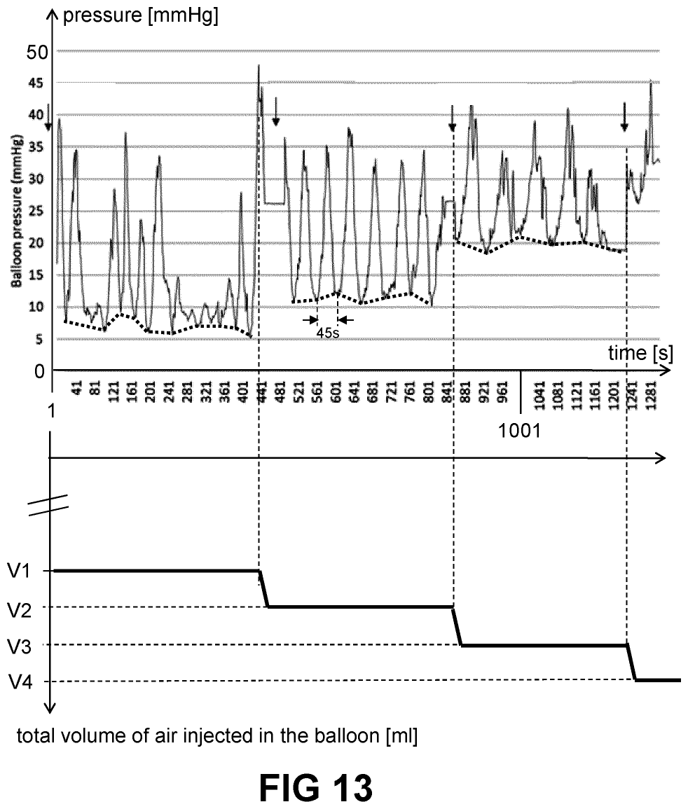

FIG. 10(a) shows a graph with five curves obtained from experiments using a balloon catheter as described in FIG. 5 above having a nominal volume of 150 ml. Each curve shows a number of data points which are interconnected, and each data point corresponds to a maximum pressure measured in a predefined time window. More specifically, FIG. 10 shows:
(i) a pressure curve (white circles) measured when the balloon is situated on a table or bench, and is stepwise being inflated in steps of 5 ml,
(ii) two pressure curves (black circles) measured when the balloon is situated in the stomach of two Healthy Volunteers (HV-1 and HV-2), and is stepwise being inflated in steps of 30 ml (from 0 to 120 ml) and then further in steps of 10 ml (from 120 m to 150 ml), and showing
(iii) two pressure curves (black squares) measured when the balloon is situated in the esophagus of two Healthy Volunteers (HV-1 and HV-2), and is stepwise being inflated in steps of 10 ml, until the Volunteers could no longer tolerate the distension (in the example: around 45 mmHg for one volunteer, and around 75 mmHg for the other volunteer).

Based on these experiments, the inventors came to the insight that a balloon with the above described characteristics yields a distinctly different pressure profile depending on where it is being inflated, e.g. (i) on a bench, (ii) in a stomach, and (iii) in the esophagus. The experiments seem to suggest that the measured balloon pressure not only depends on the inserted volume, but also depends on space constraints, e.g. on the diameter of the space where the balloon is situated. They decided to further investigate this hypothesis.

For completeness, it is noted that six points A, B, C, D, E, F are indicated on one of the curves with black circles. The skilled reader may wonder why point A (when the balloon contained 30 ml) can have a higher value than point B (when the balloon contained 60 ml), etc. FIG. 10(b) shows the raw pressure measurement data from which the points A, B, C, D, E, F are derived. As can be seen, the pressure in point A (at a volume of 30 m) was due to a short peak, which was indeed higher than the maximum pressure in point B (at a balloon volume of 60 ml), etc. This may for example occur when the person coughs or physically moves. The time durations at each pressure level were not exactly the same, but this example shows that the “maximum pressure in a given time window” is very sensitive to short peaks or outliers.

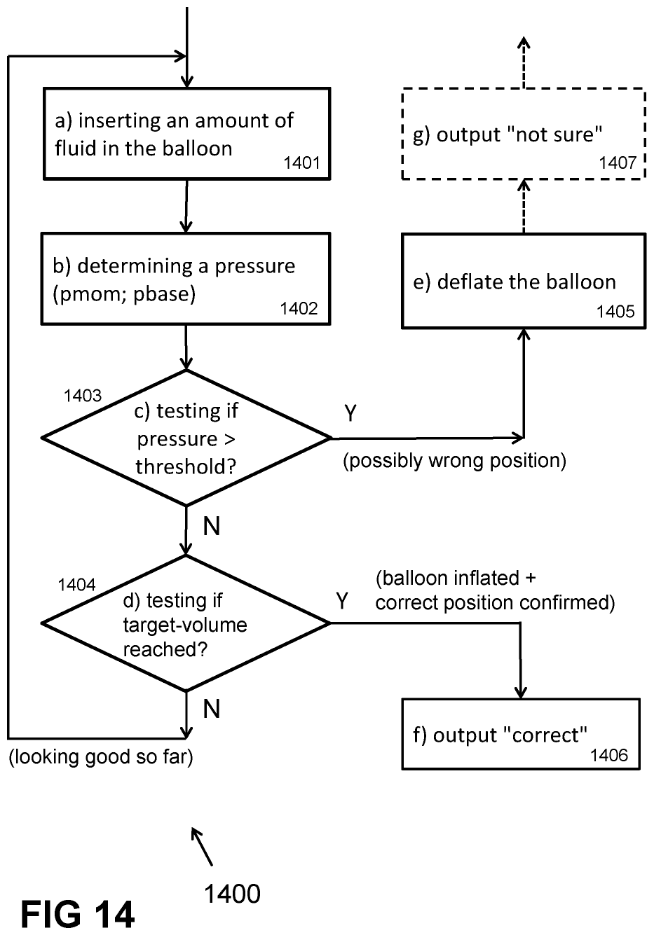

FIG. 11 shows a graph with three pressure versus volume curves obtained from experiments using a balloon catheter as shown in FIG. 5 having a nominal volume of 150 ml, showing:
i) a pressure curve (white circles) measured when the balloon is situated on a table or bench and is stepwise being inflated in steps of 5 ml,
ii) a pressure curve measured when the balloon is situated inside a “dummy trachea” (or a bench model of a trachea) having an inner diameter of about 15 mm (black circles),
iii) a pressure curve measured when the balloon is situated inside a dummy trachea having an inner diameter of about 22 mm (black squares), and
iv) a pressure curve measured when the balloon is situated inside a dummy trachea having an inner diameter of about 28 mm (black triangles).

The experimental findings described in this figure were obtained using plastic tubes of 15, 22 and 28 mm as a model for the trachea, and are assumed to represent the pressures measured in an actual human trachea of a corresponding diameter. It is known that a human trachea can have various inner diameters of about 2 cm, depending on age, sex, length and other biological variations.

These experiments confirm that the measured balloon pressure is close to zero (e.g. clearly lower than 20 mmHg) as long as the volume inserted into the balloon causes the balloon to have a diameter smaller than the inner diameter of the tube/trachea, and that the balloon pressure suddenly, and almost linearly increases with each step of 5 ml additional air injection, from the moment when the balloon diameter is equal to the inner diameter of the tube/trachea.

As can also be seen, the steepness or slope of the curves (Δp/ΔV) for a pressure lower than 100 mmHg is a value of about 16-21 for a diameter of 28 mm, and is about 24-27 for a diameter of about 22 mm, and is about 40-54 for a diameter of about 15 mm. Assuming that all human tracheas (of adults) have an inner diameter from 15 mm to 28 mm, and taking into account that this diameter is a priori unknown, it can be seen that, if this particular balloon would inadvertently be situated in the trachea (instead of in the stomach), and would be stepwise inflated in steps of 5 ml, the pressure will at some point suddenly start to increase very steeply beyond the value of 20 mmHg, and/or beyond the value of 30 mmHg, and/or beyond the value of 40 mmHg, typically by a pressure increase of about 16 to 40 mmHg at each step.

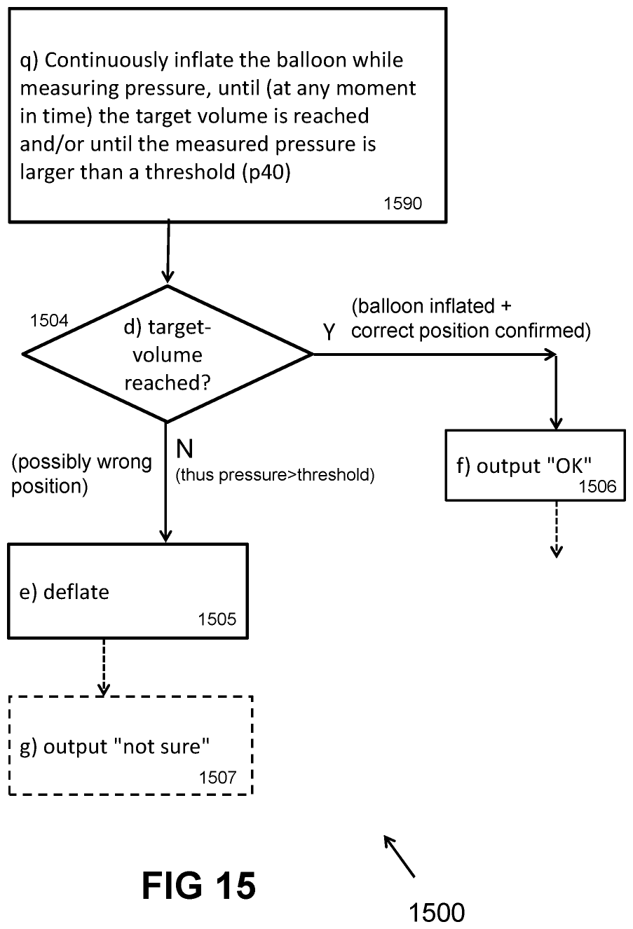

FIG. 12 shows a graph illustrating balloon pressure (vertical axis pointing upwards) and inserted volume of air (vertical axis pointing downwards) versus time (horizontal axis) of a balloon having a nominal volume of 150 ml and a nominal diameter of 4.0 cm, obtained from a bench model of a trachea having an inner diameter of 22 mm. This curve simulates balloon pressure that would be measured in case the balloon would inadvertently be situated in a trachea (instead of inside a stomach), when predefined volumes of air (e.g. 10 ml per step) are injected in the balloon in a stepwise manner: V1=60 ml, V2=70 ml and V3=80 ml, and the person would try to breath through the trachea. In this test, a person actually tried to breath through the tube to simulate the effect breathing would have on the balloon pressure. More specifically, the person periodically inhaled (causing a small drop of the balloon pressure) and then exhaled (causing the balloon pressure to restore). As can be appreciated from the pressure tracing, breathing causes “negative pressure peaks” of a relatively small amplitude (in the example: only about 5 mmHg peak-to-peak or less), and the amplitude decreases with increasing balloon volume and with increasing pressure. It was concluded that for the problem at hand, breathing does not significantly influence the balloon pressure, and can therefore be safely ignored.

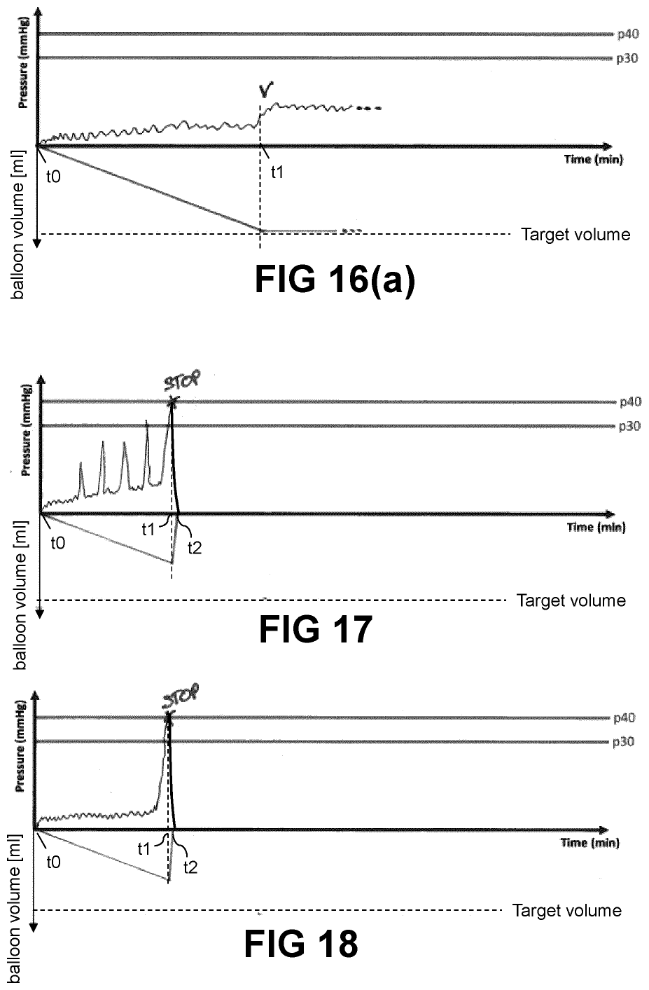

FIG. 13 shows the results of another test. This graph illustrates balloon pressure (vertical axis pointing upwards) and inserted volume of air (vertical axis pointing downwards) versus time (horizontal axis) when the balloon is positioned in the esophagus of a healthy test person. Predefined volumes of air were injected in the balloon in a stepwise manner, resulting in a total volume V1=10 ml, V2=20 ml, V3=30 ml and V4=40 ml. It can be appreciated from this figure that the balloon pressure (when the balloon is situated in the esophagus) varies significantly, even if only a very small amount of air is inserted into the balloon (e.g. a volume in the range from 10 ml to 40 ml). More specifically, and quite unexpectedly, a plurality of pressure peaks were observed, often having an amplitude larger than 15 mmHg, even up to 30 mmHg. These peaks are believed to be caused by reflex-swallowing, which is mostly an involuntary reflex. It was found that, for a given balloon, the peaks are typically larger when the balloon is situated in the esophagus, as compared to the peaks obtained from said balloon, when situated inside the stomach.

As indicated by the dotted curve segments, a baseline or minimum pressure increases stepwise with every inflation step (about 5 mmHg for the first segment, about 10 mmHg for the second segment, about 20 mmHg for the third segment, etc.). It was found that stepwise inflation of a balloon will stepwise distend the esophagus wall thereby increasing the baseline pressure, and evoking swallow reflexes. Interestingly, many of these reflexes cause the balloon pressure to be temporarily larger than about 30 to 40 mmHg.

The experiments described above confirmed the inventors' hypothesis that it is indeed possible to determine or verify or establish that the balloon is (or is not) positioned inside the stomach, mainly based on pressure measurements in relation to volume of air inserted into the balloon, but also showed that “time” may be an interesting parameter to take into account, e.g. by using a maximum pressure over a time-window, or in the form of a baseline pressure, or using a “sliding minimum pressure”. While not the primary focus of the present invention, it may even be possible to determine whether the balloon is situated in the trachea, in the esophagus or in the stomach, but only the latter is relevant for the present invention.

Taking into account that enteral feeding techniques exists already for more than a century, and taking into account the costs and burden for scanning by means of X-rays, and considering the severe consequences of misplacement, it is simply amazing that a solution based on volume and pressure and optionally also on time, without requiring magnets, pH measurements, electrical impedances, etc. could be found.

From the experiments described above, it can also be understood that it is possible to come up with more than one single algorithm for determining whether or not the balloon is situated inside the stomach, inter alia because the balloon can be inflated in many different ways: continuously, stepwise, monotonically, etc.; and because the pressure can be measured or processed in various ways, e.g. minimum pressure, maximum pressure, average pressure, instantaneous pressure, baseline pressure, time-moving-average, time-moving minimum, etc. The inventors will propose a general algorithm (see FIG. 14) and have worked out a few specific methods (see e.g. FIG. 15 and FIG. 19), but the present invention is not limited to these specific examples, and the skilled person having the benefit of the present disclosure, can easily find alternatives which fall within the scope of the general algorithm of FIG. 14. However, before describing these specific embodiments, first the general method will be described. It is pointed out in this respect that it is not absolutely required that a final decision about whether or not the balloon is situated in the stomach has to be taken after a single attempt to inflate the balloon. Indeed, in some cases the outcome of the algorithm is undecided, and the balloon is deflated as a precaution, to cause no harm to the patient. It is allowed that multiple attempts are made to inflate the balloon, with or without moving the balloon catheter prior to starting the inflation. It is of utmost importance, however, that if the algorithm decides that the balloon is indeed situated in the stomach, that this decision is highly reliable.

While the experiments described above were performed using a balloon having a nominal volume of 150 ml, the present invention is of course not limited to systems and methods using a balloon of 150 ml, and will also work for balloons having similar characteristics, as already described above (in relation to FIG. 1 to FIG. 9).

FIG. 14 shows a flowchart of a general method of assessing or determining whether a balloon 133 of a balloon catheter 130, and thus also the catheter which is fixedly connected to the balloon, is positioned in a stomach 203 of a person 210 (e.g. a living person), as proposed by the present invention. The method 1400 comprises the following steps:
a) inserting 1401 an amount of fluid in the balloon 133; e.g. inserting or injecting a predefined amount (e.g. 1 ml, or 2 ml, or 2.5 ml, or 5 ml, or 10 ml) of air inside the balloon;
b) determining 1402 a pressure inside the balloon 133; e.g. determining an instantaneous or a momentary pressure “pmom”, or a minimum pressure “pmin” or a maximum pressure “pmax” or an average pressure “pavg” over a certain time-period, or a baseline pressure “pbase” using a sliding window;
c) testing 1403 if the determined pressure is larger than a threshold (e.g. a first predefined threshold value “p40”, or a second predefined threshold value “p30”, or a dynamic threshold value “pdyn”, which will be explained further, see e.g. FIG. 16(a) to FIG. 18, FIG. 16(b), FIG. 20 to FIG. 22, and FIG. 24 to FIG. 26);
and if an outcome of this test is true, e) deflating 1405 the balloon 133 and optionally g) outputting a signal that the algorithm is undecided (i.e. is “not sure”) about the placement of the balloon;
otherwise continuing with step d) testing (implicitly or explicitly) 1404 if a predefined target volume (e.g. the nominal volume of that particular balloon) is inserted into the balloon 133;
and if an outcome of this test is true (meaning that the predefined target volume is injected in the balloon), outputting 1406 a signal indicative of correct placement of the balloon;
otherwise (meaning that the predefined target volume is not yet injected in the balloon) going back to step a), and repeating the steps above, until the determined pressure is larger than the threshold in step c), or until the target volume is inserted in the balloon in step d).

While not explicitly shown in FIG. 14, preferably the balloon is completely empty before starting step a), and it is preferably also verified that the balloon does not leak, and the balloon catheter is inserted into the patient (e.g. via the nose or via the mouth) in known manners, e.g. by inserting a certain length of the catheter into the patient, but preferably without using magnets, and preferably without using an endoscope or the like, simply based on the balloon catheter alone.

As will become clear further, if it is found in test c) that the pressure determined in step b) is larger than said threshold, this could mean (but does not necessarily mean) that the balloon catheter is not positioned in the stomach, but may be incorrectly positioned e.g. in the trachea or in the esophagus. In order to reduce potential discomfort or potential harm for the patient as much as possible, the balloon is deflated 1405 in step e), as a matter of precaution. Optionally also a signal is provided 1407 in this case, indicating that the balloon catheter may be incorrectly positioned. This is an indication to the Heath Care Provider that the balloon catheter may need to be repositioned. The Health Care Provider may optionally reposition the balloon catheter 130, and may restart the process, e.g. by using the input means 141 (see FIG. 1), e.g. by pushing a button, by pressing a key of a keyboard, by clicking a mouse, by touching a touch-panel, or in another suitable manner.

If the outcome of test d) is that the nominal volume of the particular balloon being used is inserted or injected inside the balloon (e.g. 150 ml in case the same balloon is used as was used in the experiments of FIG. 10 to FIG. 13), then it is decided that the balloon is indeed correctly positioned (i.e. is positioned in the stomach).

By choosing an appropriate value for the threshold (or thresholds) in step c), the reliability or confidence level of the method can be at least 95%.

In a variant, the order of the steps c) and d) is reversed.

In an embodiment, step c) and step d) are performed simultaneously.

In an embodiment, step a) and step b) are performed simultaneously.

In an embodiment, step a) and step b) and step c) are performed simultaneously.

In an embodiment, step a) comprises: controlling a pump, e.g. an air pump 113 illustrated in FIG. 1.

For completeness it is mentioned that the pump may be a volumetric pump or a syringe pump, or another type of pump. In an embodiment, the volume provided by the pump is proportional to the rotation speed of the pump, although that is not absolutely required. It is also possible to insert a specific amount of fluid using a pump with a predefined flow-rate and by operating the pump with an accurate timing. It is also possible to insert a specific amount of fluid using a pump with an unknown flow-rate, but by measuring the flow rate in the channel between the pump and the balloon, and by operating the pump with an accurate timing based on the measured flow rate. The system may further comprise a flowmeter. These aspects are well known in the art, and hence need not be explained in more detail here.

The method of FIG. 14 may also be formulated as follows: a method of determining or testing or evaluating or verifying or establishing whether a balloon of a balloon catheter is positioned in a stomach of a person (e.g. a living person), comprising the following steps:
a) gradually (e.g. continuously or stepwise or incrementally or intermittently) inflating 1401 the balloon and determining (e.g. continuously or periodically or repeatedly determining) 1402 a pressure (e.g. a momentary pressure; a maximum or minimum or average pressure over a certain time window; a moving minimum or moving average or moving maximum pressure over a sliding time-windows) inside the balloon until at least one of the following conditions is satisfied:
   i) the predefined nominal volume is inserted in the balloon, or
   ii) the determined pressure is larger than a threshold (e.g. a predefined threshold, or a dynamically adjustable threshold);
b) testing 1403 whether the determined pressure is, or was larger than the threshold;
and if an outcome of this test is true, deflating 1405 the balloon, and optionally providing 1407 a signal that the balloon catheter may be incorrectly positioned (i.e. that the outcome is undecided);
and if an outcome of this test is false, providing 1406 a signal indicative of correct positioning of the balloon catheter.

The balloon catheter 130 comprises a catheter and an inflatable balloon 133 fixedly connected to the catheter. The balloon has a predefined “nominal volume”.

While not explicitly shown, it is also possible to show a graphical representation of the measured pressure or a filtered version thereof, e.g. in a pressure versus volume representation, or in a pressure versus time representation, for example on a graphical display, e.g. an LCD display, or a touchscreen, or the like. By doing so, not only does the operator (or Health Care Provider) get an idea of the progress during the process, but he or she may also get a better indication of how quickly the pressure threshold(s) was reached, and/or about the margin between the determined pressure and the applicable threshold value(s). Indeed, if the threshold was reached only relatively late, e.g. when the balloon was already inflated to about 95% of its nominal volume, chances are high that the balloon may still be located in the stomach, and that (complete) repositioning may not be required. In contrast, if the threshold pressure was reached very early, e.g. when only 10 ml or 20 ml was inserted into the balloon, this may be a strong indication that the balloon catheter is very likely not situated in the stomach. In both cases, however, the procedure has to be restarted to ascertain that the balloon is positioned in the stomach before enteral feeding is supplied to the patient.

Instead or in addition to showing a graphical representation of the pressure curve, it is also possible to show other data, such as a progress bar showing a ratio of the volume already inserted into the balloon and the nominal volume; and/or the time lapsed since the start of the procedure; and/or the maximum pressure value measured and the time when this occurred, etc.

In some embodiments of the present invention, the system may provide additional information in step g), such as textual or audible information, e.g. containing a suggestion of what to do next, e.g. an indication or suggestion that the balloon catheter may need to be repositioned or has to be repositioned.

In some embodiments of the present invention having a food pump, the system may have provisions that the feeding pump cannot be started unless it was successfully determined that the balloon catheter is correctly positioned. In this way, the risk of human errors can be reduced.

FIG. 15 shows a flowchart of a first specific method 1500 of determining or testing or evaluating or verifying or establishing or assessing whether a balloon 133 of a balloon catheter 130 having the properties described above, is positioned in the stomach of a person, referred to herein as “the fast method”, which is a special case of the method 1400 of FIG. 14. The method 1500 comprises the following steps:
q) continuously inflating 1590 the balloon while measuring balloon pressure, until (at any moment in time) the nominal volume is reached and/or until the measured pressure is larger than a predefined threshold (e.g. a predefined threshold level p40, or a dynamically adjusted threshold level p40a, p40b which is dependent on the volume injected);
d) after step a) has stopped, testing 1504 (directly or indirectly) if the nominal volume is reached,
and if the outcome of this test is true, (meaning that the balloon was inflated in one go without the pressure at any moment in time being larger than the applicable threshold), providing 1506 a signal that the balloon 133 is positioned in the stomach;
and if the outcome of this test is false, (meaning that the measured pressure at some point in time was larger than the applicable threshold), deflating 1505 (e.g. completely deflating) the balloon, and optionally providing 1507 a signal that the balloon may not be situated in the stomach. As mentioned above, this does not necessarily mean that the balloon is not situated in the stomach, but it means that the algorithm is not sure and that the position is undecided.

It is noted that step q) of method 1500 corresponds to steps a) and b) and c) of method 1400.

In embodiments where the threshold is not a single predefined value, step q) may further comprise: determining the applicable threshold depending on the time since the start, or depending on the number of the iteration, or depending on the cumulative amount of fluid injected into the balloon.

In a variant of this method (not shown), step d) is replaced by testing if the measured pressure was at any moment in time, larger than the applicable (e.g. predefined) threshold value (e.g. by testing a memory location, or a latch or another memory element);

and if the outcome of this test is false, (meaning that the measured pressure was not larger than the threshold), providing 1506 a signal that the balloon is positioned in the stomach;

and if the outcome of the test is true, (meaning that the measure pressure was larger than the applicable threshold at a moment in time), deflating 1505 the balloon, and optionally providing 1507 a signal that the balloon may not be situated in the stomach.

In an embodiment, step a) comprises starting a pump, and letting the pump operate until one or both of said conditions is reached.

In both methods (the method shown in FIG. 15 and the variant), step a) may comprise: starting a pump (e.g. an air pump), and letting the pump operate until one or both of said conditions is satisfied.

The principles of this algorithm will be better understood by means of the examples shown in FIG. 16(a) to FIG. 18 and FIG. 16(b).

FIG. 16(a) shows an example of how the pressure (vertical axis pointing upwards) inside the balloon may vary as a function of time (horizontal axis), when applying the method of FIG. 15. Also shown is the (total) volume of air inside the balloon (vertical axis pointing downwards). In the example of FIG. 16(a) the balloon is correctly positioned (i.e. in the stomach), and there are no gastric contractions.

At the start of the test, the balloon is empty (volume=0 ml) and the measured pressure is 0 mmHg. At time t0, step q) is started, meaning that the balloon is being inflated, continuously or quasi-continuously, meaning: gradually, without a pause larger than 0.5 seconds (e.g. using a stepper motor), while the pressure inside the balloon is being measured, continuously or quasi-continuously.

The expression “quasi-continuously” can mean e.g. periodically at a frequency of at least 0.5 Hz, or at least 1 Hz, or at least 2 Hz, or at least 5 Hz, or at least 10 Hz, or can mean: taking at least one pressure sample for every injection of at most 10 ml, or at most 5 ml, or at most 2 ml or at most 1 ml.

In an embodiment, the measurement sampling rate depends on the inflation rate. The inflation rate may be a value in the range from 2.5 ml/sec to 40 ml/sec, or in the range from 2.5 ml/sec to 30 ml/sec, or in the range from 4 ml/sec to 25 ml/sec, or in the range from 5 ml/sec to about 20 ml/sec, or in the range from 5 ml/sec to about 15 ml/sec, e.g. equal to about 10 ml/sec.

In a preferred embodiment, the inflation rate may be about 10 ml/sec, and the sampling rate may be at equal to about 5 Hz or 10 Hz.

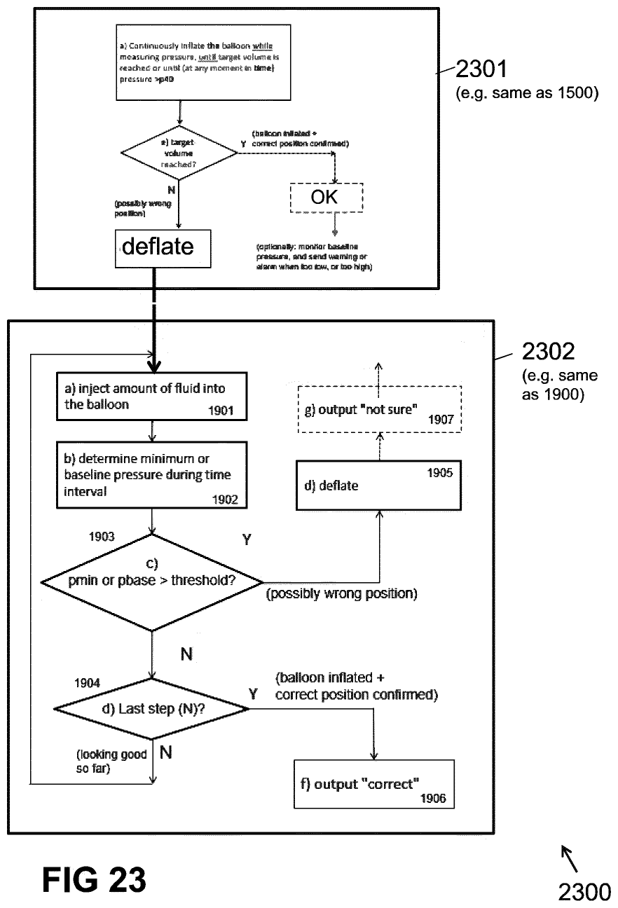

Two predefined threshold values p30 and p40 are shown in the drawing, for reasons which will become clear further (as a comparison), but in the method of FIG. 15 only one threshold value, namely the threshold value labelled “p40” is used. The value of p40 is a predefined value, for example a constant value in the range from about 21 mmHg to about 99 mmHg (i.e. from about 2800 Pa to about 13199 Pa); or from about 26 mmHg to about 79 mmHg (i.e. from about 3466 Pa to about 10532 Pa); or from about 31 mmHg to about 49 mmHg (i.e. from about 4133 Pa to about 6533 Pa). This value may be fixed, independent of the particular patient, or may be chosen dependent on the patient, e.g. dependent on the patient's sex (male or female), age, length, weight, etc., for example using a look-up table. In any case, for the method of FIG. 15, at each moment in time, the value of the threshold “p40” is a constant pressure value, for example 40 mmHg. In the examples of FIG. 16(a), FIG. 17 and FIG. 18, the threshold value “p40” is chosen to be a predefined constant, independent of the volume inserted, for simplicity.

During the test, the balloon is gradually being inflated, the pressure is being measured, the measured pressure is compared with the applicable threshold value denoted as “p40”, and the inserted volume is compared with the nominal volume of the particular balloon being used, until at least one of the end conditions is met.

Since in the example of FIG. 16(a) the balloon is positioned in the stomach, and the stomach has no contractile activity, and there was no other reason for a pressure increase (e.g. coughing or movement of the patient), the pressure in the balloon varies slightly with breathing, but remains well-below the p40 threshold value. The balloon can thus be completely inflated according to the flowchart described in FIG. 15. When the nominal volume is inserted in the balloon at time t1, stap q) has reached an end condition, and a signal will be provided to notify the operator that the balloon is indeed positioned in the stomach.

FIG. 16(a) illustrates an ideal case, in which the balloon can be inflated in one go. It can be appreciated that the method of FIG. 15, if it succeeds, is extremely fast, as compared to for example an X-ray scan. It is estimated that a successful outcome in one go will probably occur in about 80% of the cases.

FIG. 17 shows another example of how the pressure and the volume of the balloon may vary as a function of time, when applying the method of FIG. 15. In the example of FIG. 17 the balloon is also positioned in the stomach, but there are relative strong gastric contractions.

At the start of the test, the balloon is empty (volume=0 ml) and the measured pressure is 0 mmHg. After time t0, the balloon is continuously or quasi-continuously being inflated while the pressure inside the balloon is continuously or quasi-continuously being measured.

As in this example the balloon is positioned in the stomach and the stomach has contractile activity, the pressure in the balloon will vary because of breathing (small ripple, typically about 15 times per minute) but mainly due to stomach contractile activity (relatively large peaks, typically about 3 times per minute). As stomach contractions can induce relative high balloon pressure peaks it is possible that, even though the balloon is correctly positioned, the threshold pressure (p40) is reached at time t1, and as a consequence of the flowchart described in FIG. 15 the balloon is deflated, e.g. immediately after detection that the pressure has reached the threshold, causing the pressure to drop instantly too. As described above, the user or operator may be notified that the assessment failed, and/or that the balloon may not be positioned in the stomach.

In the example of FIG. 17, the balloon is empty again at time t2, and the pressure has reached 0 mmHg. If the operator decides that no repositioning is required, a new attempt to ascertain that the balloon catheter was positioned in the stomach after all, can then be started.

For completeness it is noted that, even though five gastric peaks are shown in FIG. 17, this does not imply that the inflation of the balloon to its nominal volume requires more than 3 or 4 minutes. Indeed, the balloon may be inflated faster, but the example of FIG. 17 wanted to illustrate that some peaks may not reach the threshold pressure while other peaks may.

FIG. 18 shows another example of how the pressure and the volume of the balloon may vary as a function of time, when applying the method of FIG. 15. In the example of FIG. 18 the balloon is inadvertently situated in the trachea.

At the start of the test, the balloon is empty (volume=0 ml) and the measured pressure is 0 mmHg. At time t0, the balloon is continuously or quasi-continuously being inflated while the pressure inside the balloon is continuously or quasi-continuously being measured.

As in this example the balloon is inadvertently positioned in the trachea, the balloon pressure varies with breathing as long as the balloon does not occlude the trachea. As soon as the balloon occludes the trachea, the pressure rapidly increases (as also illustrated in FIG. 11) until the threshold pressure (p40) is reached at time t1, and as a consequence of the flowchart described in FIG. 15 the balloon is deflated, and the user may be notified accordingly. In the example, the balloon is empty again at time t2.

FIG. 16(a), FIG. 17 and FIG. 18 illustrate the method of FIG. 15 using a single fixed threshold value, but the present invention is not limited thereto, and embodiments using at least two fixed threshold values are also contemplated.

FIG. 16(b) is a variant of FIG. 16(a) illustrating a variant of the method of FIG. 15, in which two threshold values p40a, p40b are used. In the specific example shown, a first threshold value p40a is used as long as the volume inserted into the balloon is smaller than a predefined volume (in the example 50% of the nominal volume, e.g. 75 ml), and a second threshold value p40b (larger than the first threshold p40a) is used when the volume inserted into the balloon is larger than said predefined volume. Step a) of FIG. 14 or step q) of FIG. 15 may further comprise: “determining the applicable threshold value” e.g. as a function of the inserted volume so far, or as a function of time, or as a function of the iteration number, before performing the actual comparison. In the example of FIG. 16(b) this determination can be as simple as selecting one of two predefined values, depending on whether the inserted volume is smaller or larger than a predefined volume (e.g. 75 ml), but of course, the present invention is not limited hereto. For example, it is possible to use more than two values, for example at least three values, or more than three values. The “ceiling function” would then look like a staircase with multiple steps. When using the method of FIG. 15 where the inflation is a continuous or quasi-continuous function of time, the threshold value may also be determined as a continuous function of time. In case the inflation occurs stepwise, e.g. in steps of 5 ml, the threshold value may be determined using a look-up table, where the threshold may be adjusted for each step. This would require for example a look-up table of 30 pressure values for a balloon having a nominal volume of 150 ml, which is inflated in steps of 5 ml. This is very easy to implement, but allows to delimit or delineate the region of allowed pressure-versus-volume couples with much finer granularity than the case illustrated in FIG. 16(a) with a single threshold value, or the case illustrated in FIG. 16(b) with two threshold values. As an example, if the curves of FIG. 10 would be representative for a large population, it would for example be possible to detect that the balloon is situated in the trachea already after injecting 10 ml into the balloon, e.g. by setting the pressure threshold value (i.e. the maximum allowed pressure) corresponding to a value of 10 ml for example to a value in the range from 20 to 25 mmHg.

In an even more sophisticated implementation, the method of FIG. 14 or FIG. 15 would not only use a plurality of threshold values, for example stored in a 1-dimensional look-up table containing for example 30 values (e.g. one value for each step of 5 ml; 150 ml=30*5 ml), but may use a two-dimensional look-up table with a plurality of columns, e.g. depending on sex, age, weight, length of the patient, or combinations hereof.

FIG. 19 shows a flow chart of a second specific method 1900 of determining or testing or evaluating or verifying or establishing or assessing whether a balloon of a balloon catheter is positioned in the stomach of a person, referred to herein as “the slow method”, which is also a special case of the method 1400 of FIG. 14. The method 1900 comprises the following steps:
a) injecting 1901 a discrete amount of fluid (e.g. air) into the balloon (e.g. in steps from 1 ml to 20 ml, e.g. in steps of about 2 ml or 5 ml or 10 ml in each step);
b) determining 1902 a minimum pressure (pmin) or a baseline pressure (pbase) during a time interval (e.g. a predefined time interval labelled “T30”, e.g. 30 seconds)
c) testing 1903 whether the minimum or baseline pressure is larger than a threshold value (e.g. a predefined threshold value, or a dynamically adjusted threshold value);
and if the outcome of this test is true, e) deflating 1905 the balloon, and optionally outputting a signal for indicating that the balloon may be incorrectly positioned;
and if the outcome of this test is false, continuing with step d) of testing 1904 whether this was the last step (referred to herein as step “N”);
and if the outcome of this test is true, f) providing 1906 an output signal that the balloon is correctly positioned;
and if the outcome of this test is false, going back to step a).

Many implementations and many variants of this method are envisioned.

In an embodiment, step b) of each iteration is performed only after step a) of that iteration is completed.

In another embodiment, step b) is at last partially overlapping with step a), for example when using a “sliding minimum pressure”.

The last step “N” is the step after which the balloon is filled to its “target volume” (also referred to herein as its “nominal volume”).

In an embodiment, the amount of fluid to be injected in step a) is not constant, but may vary with the number of the step.

In a simple embodiment, step c) may comprise: comparing the minimum or baseline pressure to a single, predefined threshold value “p30”, applicable for all the steps.

In another embodiment, step c) may comprise: comparing the minimum or baseline pressure to a first threshold value “p30a” for step 1 to N−1, and comparing the minimum or baseline pressure to a second threshold value “p30b” for the last step N, during which the balloon will reach its nominal value.

In yet another embodiment, step c) may comprise: comparing the minimum or baseline pressure to a first threshold value “p30a” for step 1 to N−2, and comparing the minimum or baseline pressure to a second threshold value “p30b” for the last two steps (N−1 and N).

It is of course also possible here to use more than two threshold values, e.g. at least three, or at least four threshold values, or a one-dimensional array of threshold values, e.g. a different one for each separate step.

And as was explained above, it is also possible here to use a two-dimensional look-up table with a plurality of columns, e.g. depending on sex, age, weight, length of the patient, or combinations thereof. Furthermore, it is not required that the same amount of fluid is inserted in each step. Also the amount of fluid itself may depend on the step. These amount values may be stored in a one-dimensional array, or a two-dimensional array with multiple columns.

In an embodiment, the time intervals (of step b) may have a predetermined duration, e.g. as a value in the range from about 10 seconds to about 60 seconds, or in the range from about 20 s to about 50 s, e.g. equal to about 25 s, or equal to about 30 s, or equal to about 35 s, or equal to about 40 s, or equal to about 45 s. In other embodiments, the time intervals may be dynamically determined, e.g. to substantially coincide with the moments of start and stop of gastric contraction peaks.

The main difference between the “slow” method of FIG. 19 and the “fast” method of FIG. 15 is that the inflation of the method of FIG. 19 occurs in several discrete steps, separated from each other by time intervals during which the volume inside the balloon is kept fixed, and during which time intervals a minimum pressure or a baseline pressure is determined which is then compared to a threshold value.

The main disadvantage of the method 1900 (as compared to the “fast method” of FIG. 15) is that it requires more time. The main advantage of this method is that the minimum pressure or baseline pressure allows to filter out or reduce the gastric contraction peaks.

The principles of this algorithm will be better understood by means of the examples shown in FIG. 20 to FIG. 22.

FIG. 20 shows an example of how the pressure and the volume of the balloon may vary as a function of time, when applying the method of FIG. 19. In the example of FIG. 20 the balloon is positioned in the stomach, and there is no gastric activity.

At the start of the test, the balloon is empty (volume=0 ml) and the measured pressure is 0 mmHg. In this illustration the balloon is stepwise inflated in three steps (N=3), for illustrative purposes. After each inflation step the balloon volume is kept constant for a predefined period of time (e.g. 60 seconds). During this period in which the balloon volume is kept constant, a minimum pressure is determined, or a “moving minimum” is determined over a sliding window having a duration from 5 seconds to 60 seconds (e.g. equal to about 10 s, or equal to about 20 s, or equal to about 30 s, or equal to about 40 s, or equal to about 50 s), or another type of baseline pressure.

In the example shown in FIG. 20, it is assumed that the balloon is positioned in the stomach, and that there is only one single threshold value “p30” to be considered, applicable for all the steps, including the last step. In practice there may be more than three steps, but only three steps are shown here to keep the drawing and the explanation simple. The signal with the ripple represents the actual pressure measurement, the smooth line underneath shows a moving minimum pressure of this curve over a time window of 10 seconds.

As the balloon in this example is positioned in the stomach, the moving minimum pressure in the balloon remains well-below the p30 threshold value, and the balloon can be completely inflated to its nominal volume, using the algorithm described in FIG. 19. The method will output a signal that the balloon is correctly positioned. As can be seen, there is a relatively large margin between the pressure and the threshold. The larger this margin, the larger the confidence level that the balloon is indeed positioned in the stomach.

FIG. 21 shows another example of how the pressure and the volume of the balloon may vary as a function of time, when applying the method of FIG. 19. In the example of FIG. 21 the balloon is positioned in the stomach, and there is gastric activity.

In this example, it is assumed that the algorithm uses two threshold values: “p30a” applicable for step N=1 and 2, and “p30b” applicable for the last step (N=3).

The balloon is again stepwise inflated, and after each inflation step the balloon volume is kept constant for a predefined period of time (e.g. 60 seconds), during which period a minimum pressure or a “sliding minimum” over a predefined time window having a duration from 5 to 60 seconds (e.g. equal to about 10 s, or equal to about 20 s, or equal to about 30 s, or equal to about 40 s, or equal to about 50 s) is calculated.

As in this example the balloon is positioned in the stomach, the momentary pressure values may be larger than the threshold values “p30a” or “p30b”, but in the method 1900 of FIG. 19 it is not the momentary value that needs to be compared with the threshold values, but the baseline value. As can be seen from the example, the baseline remains well-below the first p30a threshold pressure during step N=1 and step=2, and remains well below the second threshold p30b during step N=3. The balloon can be completely inflated to its nominal volume, according to the flowchart described in FIG. 19. The method will output a signal that the balloon is correctly positioned

FIG. 22 shows another example of how the pressure and the volume of the balloon may vary as a function of time, when applying the method of FIG. 19. In the example of FIG. 22 the balloon is inadvertently situated in the trachea.

The balloon is again stepwise inflated, and after each inflation step the balloon volume is kept constant for a predefined period of time (e.g. 60 seconds), during which period a minimum pressure or a “sliding minimum” over a predefined time window having a duration from 5 to 60 seconds (e.g. equal to about 30 s) is calculated.

As in this example the balloon is mispositioned in the trachea, the moving minimum value will exceed the “p30” threshold long before the nominal volume is inserted into the balloon. As a consequence, the balloon will be deflated according to the algorithm described in FIG. 19, and a signal or a message may be provided to the operator to indicate that the balloon may be mispositioned.

When discussing FIG. 14, it was mentioned that “additional information” may be provided to the operator. In some embodiments, a “confidence level” may be provided as extra information. For example, in the case of FIG. 20 and FIG. 21, the confidence level that the balloon is indeed positioned in the stomach is very high, e.g. larger than 99%, because the difference between the minimum pressure and the threshold p30 is very large (e.g. larger than 20 mmHg). In contrast, in FIG. 22, the confidence level that the balloon catheter is situated in the stomach is extremely low. The operator may use this information in his decision whether or not to reposition the catheter before starting another attempt.

FIG. 23 shows a flowchart of a third specific method of determining or testing or evaluating or verifying or establishing or assessing whether the balloon of a balloon catheter is positioned in a stomach of a person, referred to herein as “the combined method”, which is composed of the steps of the (fast) method of FIG. 15, and in case the outcome of the fast method is unsuccessful, followed by the steps of the (slow) method of FIG. 19.

Using the combination method, an operator can first try to assess whether the balloon catheter is positioned in the stomach in a very fast way, which will probably succeed in 80% of the cases, but will sometimes fail, even if the balloon catheter is positioned in the stomach. If that is the case, the operator may try the “slow method”, which will require more time, but chances of failing if the balloon catheter is correctly positioned is very low.

It is also possible to simultaneously perform some of the principles of FIG. 15 (e.g. continuous or quasi-continuous inflation, and comparing instantaneous pressure with a threshold p40) with some of the principles of FIG. 19 (e.g. determining a baseline or a “sliding minimum”, and comparing that sliding minimum with a threshold p30), and combining the outcomes of the two comparisons.

In some embodiments, the inflation is stopped and the balloon will be deflated if at least one of the threshold values is reached. Thus, in order for the algorithm to decide that the balloon is situated in the stomach, it is required that the nominal volume is inserted into the balloon AND the instantaneous pressure “pmom” was always smaller than p40 AND the baseline pressure was always smaller than p30. Such methods are further referred to herein as “mixed-type1” methods. These methods may detect a mispositioned balloon earlier, but may have a “first-time pass-rate” lower than 80%.

In other embodiments, the inflation is stopped and the balloon will be deflated only if both threshold values are reached. Thus, in order for the algorithm to decide that the balloon is situated in the stomach, it suffices that the nominal volume is inserted into the balloon AND (the instantaneous pressure pmom was always smaller than p40 OR the baseline pressure was always smaller than p30). Such methods are further referred to herein as “mixed-type2” methods. These methods may detect a mispositioned balloon somewhat later, but may have a “first-time pass-rate” larger than 80%.

The values for p30 and p40 may be different for the “mixed-type 1” and “mixed-type 2” method, and similar to what was described above, use can be made of multiple values p30a, p30b etc. and/or multiple values p40a, p40b, etc. as a function of the inserted volume.

By choosing appropriate threshold values, both “mixed-type 1” methods, and “mixed-type2” methods can be made highly reliable when they decide that the balloon is situated in the stomach.

FIG. 24 shows an example of how the pressure and the volume of the balloon may vary as a function of time, when applying a “mixed-type 1 method” or a mixed-type 2 method” described above.

In this example, a single value of p30 and p40 is used for both mixed-type methods, to keep the explanation simple. The same waveform is shown as in FIG. 16, but a baseline is added. In this example, the baseline is calculated as a “moving minimum” over a sliding time window of 30 s. Since none of the peaks reaches the threshold level p40, and since the baseline does not reach the threshold level p30, both mixed-type methods will inflate the balloon in one go, and both mixed methods will decide that the balloon is situated in the stomach.

FIG. 25 shows an example of how the pressure and the volume of the balloon may vary as a function of time, when applying a “mixed-type 2 method” described above. Also in this example, a single value of p30 and p40 is used for both mixed-type methods, to keep the explanation simple. In this example, the balloon is situated in the stomach, and there are relatively strong gastric contractions.

As described above, in the “mixed-type 2 method” the inflation will stop when both the baseline pressure is larger than a threshold p30, and the momentary peaks are larger than a threshold p40, but since the baseline pressure is always smaller than p30, the “mixed-type 2 method” will inflate the balloon in one go, and will decide that the balloon is situated in the stomach. In this example, the baseline is calculated as a “moving minimum” over a sliding time window of about 30 s.

If a “mixed-type 1 method” were applied using the same threshold values p30 and p40, the method would stop inflating (not shown) at time tx, because the momentary peak pressure is larger than p40, and the balloon would be deflated, and a signal may be provided to the operator indicating that the balloon may be mispositioned.

FIG. 26 shows an example of how the pressure and the volume of the balloon may vary as a function of time, when applying a “mixed-type 1 method” or a mixed-type 2 method” described above. Also in this example, a single value of p30 and p40 is used for both mixed-type methods, to keep the explanation simple. In this example, the balloon is situated in the trachea. The same waveform is shown as in FIG. 22, but a baseline is added. In this example, the baseline is calculated as a “moving minimum” over a sliding time window of 10 s. In the particular example shown, it is assumed that the baseline reaches the level p30 at time tx, and the instantaneous peaks reach the level p40 at time ty, slightly later than tx.

If a “mixed-type 1 method” is applied, the method will stop inflating at tx (because the baseline pressure reaches p30). If a “mixed-type 2 method” is applied, the method will stop inflating at ty (because the momentary pressure reaches p40). In this example, both methods will stop inflating the balloon, and will deflate the balloon, and may provide a signal to the operator that the balloon may be mispositioned.

In the description above, the threshold p40 (or p40a, p40b, etc.) is typically used for comparison with a momentary pressure, and the threshold values p30 (or p30a, p30b, etc.) is typically used for comparison with a minimum pressure, or a moving or sliding minimum pressure, or a baseline pressure over a time window. The time windows may have a duration from 5 seconds to 60 seconds, e.g. equal to about 10 seconds or equal to about 30 seconds.

In embodiments where only p30 is used (see e.g. FIG. 20, FIG. 22), the value of the pressure threshold p30 may be a value in the range from 15 mmHg to 39 mmHg (i.e. from about 2000 to about 5200 Pa), or from 21 mmHg to 39 mmHg, or in the range from 25 mmHg to 35 mmHg, e.g. equal to about 30 mmHg.

In embodiments where only p40 is used (see e.g. FIG. 16(a), FIG. 17, FIG. 18, FIG. 16(b)) the value of the pressure threshold p40 may be a value in the range from 21 mmHg to about 99 mmHg (i.e. from about 2800 Pa to about 13199 Pa); or from about 26 mmHg to about 79 mmHg (i.e. from about 3466 Pa to about 10532 Pa), or from 31 mmHg to 55 mmHg (i.e. from about 4133 to about 7333 Pa), or from 35 mmHg to 50 mmHg (i.e. from about 4666 Pa to about 6666 Pa), e.g. equal to about 40 mmHg (i.e. about 5333 Pa).

In embodiments where both p30 (or p30a, p30b, etc.) and p40 (or p40a, p40b. etc.) are used, (see e.g. FIG. 24 to FIG. 26), the value of the pressure threshold p30 may be a value in the range from 15 mmHg to 39 mmHg (i.e. from about 2000 to about 5200 Pa), and the value of p40 may be a value from (p30+1) mmHg to 99 mmHg (i.e. about 13199 Pa), or from (p30+5) mmHg to 55 mHg (i.e. about 7333 Pa).

While the present invention is explained and illustrated above primarily for determining whether a balloon of a balloon catheter is positioned in the stomach of an adult or a young adult, the present invention is not limited thereto, and the invention also works for determining whether the balloon of a balloon catheter is positioned in the stomach of children or infants or babies. Of course, in this case a smaller balloon will have to be used (e.g. a balloon having a nominal volume smaller than 120 ml, or smaller than 90 ml, or smaller than 60 ml; and having an outer diameter smaller than 3.0 cm, or smaller than 2.5 cm, or smaller than 2.0 cm); and in case of stepwise inflation, smaller volumes AV may have to be inserted in each step, for example at most 3 ml per step, or at most 2 ml per step; and suitable threshold levels will need to be chosen, but apart from these parameters, the same algorithms as described above can also be used. The skilled reader having the benefit of the present disclosure, can find suitable parameters for a particular balloon, e.g. using the same or similar experiments as described above.

1. A system (100) for determining whether a balloon (133) of a balloon catheter (130) is positioned in a stomach (203) of a person (210), the system (100) comprising:
said balloon catheter (130) comprising a catheter and an inflatable balloon (133) fixedly attached to said balloon catheter;
an inflation device (113) operatively connected to the balloon for selectively inflating or deflating the balloon;
a pressure sensor (114) operatively connected to the balloon for measuring a pressure inside the balloon;
an output device (142) for providing a signal to an operator;
a control unit (110) connected to the pressure sensor (114) and to the inflation device (113) and to the output device (142), and configured for performing an algorithm comprising the following steps:
a) inserting (1401) an amount of fluid in the balloon (133);
b) determining (1402) a pressure inside the balloon (133);
c) testing (1403) if the determined pressure is larger than a threshold (p40; p30),
and if an outcome of this test is true, e) deflating (1405) the balloon;
otherwise continuing with step d);
d) testing (1404) if a predefined nominal volume (Vnom) is injected in the balloon,
and if an outcome of this test is true, outputting (1406) a signal indicating that the balloon is situated in the stomach;
otherwise going back to step a), and continuing until the pressure is larger than the threshold (p40; p30) in step c), or until the nominal volume is inserted into the balloon in step e).

2. System (100) according to claim 1,
wherein step a) further comprises: determining the threshold (p40; p30) to be used in step c) as a function of the amount of fluid inserted in the balloon, or as a function of time, or as a function of an iteration number.

3. System (100) according to claim 1 or 2,
wherein step b) comprises: measuring (1402; 1502) a momentary pressure (pmom) inside the balloon (133), and considering the momentary pressure as the determined pressure; and
wherein step c) comprises: testing (1403; 1503) if the momentary pressure (pmom) is larger than a first threshold (p40).

4. System (100) according to any of the previous claims,
wherein step b) comprises measuring (1402; 1902) a plurality of momentary values of the pressure inside the balloon (133), and calculating a filtered pressure value based on said plurality of values; and
wherein step c) comprises testing (1403; 1903) if the filtered pressure value is larger than a second threshold (p30).

5. System (100) according to any of the previous claims,
wherein step b) comprises: measuring (1402; 1902) a plurality of pressure values during a predefined time period or time window, and calculating an minimum pressure, or calculating a moving minimum pressure during a predefined time-window, and considering this minimum or moving minimum as the filtered pressure value.

6. System (100) according to any of the previous claims,
wherein step b) comprises: measuring (1402; 1902) a plurality of pressure values during a predefined time period or time window, and calculating an average pressure, or calculating a moving average pressure during a predefined time-window, and considering this average or moving average as the filtered pressure value.

7. System (100) according to any of the previous claims,
wherein step a) comprises: determining an amount of fluid to be inserted, and inserting the determined amount of fluid into the balloon; and
wherein step e) comprises: summing the determined amounts of fluid and comparing the summed amount with the predefined nominal volume, or determining a number of the iteration, and comparing this number with a predefined target number.

8. System (100) according to any of the previous claims,
wherein the controller is configured for performing step a) and b) and c) substantially simultaneously.

9. System (100) according to any of the previous claims,
wherein the controller is configured for performing step a) during first time periods; and
wherein the controller is configured for performing step b) during second time periods; and
wherein the first and second time periods are at least partially overlapping.

10. System (100) according to any of the previous claims,
wherein the controller is configured for performing step a) during first time periods; and
wherein the controller is configured for performing step b) during second time periods; and
wherein the first and second time periods are non-overlapping.

11. System (100) according to any of the previous claims,
wherein the controller is configured for inflating the balloon as specified in claim 3 as a first attempt to inflate the balloon to its nominal volume without the momentary pressure (pmom) becoming larger than the first threshold (p40);
and in case the balloon was deflated in step e) of the first attempt, for inflating the balloon as specified in claim 4 or 5 or 6 as a second attempt to inflate the balloon to its nominal volume without the filtered pressure becoming larger than the second threshold (p30).

12. System according to any of the previous claims,
wherein the output device (142) comprises at least one of: a speaker, a display, an LCD display, a light emitting device, one or more light emitting diodes (LEDs), a vibration element, a buzzer, a touchscreen.

13. System according to any of the previous claims, further comprising at least one of the following features:
i) an input device (141) for receiving a command from an operator;
ii) a timer;
iii) a digital processor.

14. System according to any of the previous claims,
further comprising a mechanical valve configured for at least partially deflating the balloon in case the pressure inside the balloon is higher than a third predefined value.

15. Method (1400; 1500; 1900; 2300) of determining whether a balloon (133) of a balloon catheter (130) is positioned in a stomach (203) of a person (210), the method comprising the steps of:
a) inserting (1401) an amount of fluid in the balloon (133);
b) determining (1402) a pressure inside the balloon (133);
c) testing (1403) if the determined pressure is larger than a threshold (p40; p30),
and if an outcome of this test is true, e) deflating (1405) the balloon;
otherwise continuing with step d);
d) testing (1404) if a predefined nominal volume (Vnom) is injected in the balloon,
and if an outcome of this test is true, outputting (1406) a signal indicating that the balloon is situated in the stomach;
otherwise going back to step a), and continuing until the pressure is larger than the threshold (p40; p30) in step c), or until the nominal volume is inserted into the balloon in step e).

16. Method according to claim 15,
wherein step a) further comprises: determining the threshold (p40; p30) to be used in step c) as a function of the amount of fluid inserted in the balloon, or as a function of time, or as a function of an iteration number.

17. Method (1400; 1500) according to claim 15 or 16,
wherein step b) comprises measuring (1402; 1502) a momentary pressure (pmom) inside the balloon (133), and considering the momentary pressure as the determined pressure; and
wherein step c) comprises testing (1403; 1503) if the momentary pressure (pmom) is larger than a first threshold (p40).

18. Method (1400; 1500; 1900) according to any of the claims 15 to 17,
wherein step b) comprises measuring (1402; 1902) a plurality of momentary values of the pressure inside the balloon (133), and calculating a filtered pressure value based on said plurality of values; and
wherein step c) comprises testing (1403; 1903) if the filtered pressure value is larger than a second threshold (p30).

19. Method (1400; 1500; 1900) according to any of the claims 15 to 18,
wherein step b) comprises: measuring (1402; 1902) a plurality of pressure values during a predefined time period or time window, and calculating an minimum pressure, or calculating a moving minimum pressure during a predefined time-window, and considering this minimum or moving minimum as the filtered pressure value.

20. Method (1400; 1500; 1900) according to any of the claims 15 to 19,
wherein step b) comprises: measuring (1402; 1902) a plurality of pressure values during a predefined time period or time window, and calculating an average pressure, or calculating a moving average pressure during a predefined time-window, and considering this average or moving average as the filtered pressure value.

21. Method according to any of the claims 15 to 20,
wherein step a) comprises: determining an amount of fluid to be inserted, and inserting the determined amount of fluid into the balloon; and
wherein step e) comprises: summing the determined amounts of fluid and comparing the summed amount with the predefined nominal volume, or determining the number of the iteration, and comparing this number with a predefined target number.

22. Method (1500) according to any of the claims 15 to 21,
wherein step a) and b) and c) are performed substantially simultaneously.

23. Method (1400; 1500) according to any of the claims 15 to 22,
wherein step a) is performed during first time periods; and
wherein step b) is performed during second time periods; and
wherein the first and second time periods are at least partially overlapping.

24. Method (1400; 1900) according to any of the claims 15 to 23,
wherein step a) is performed during first time periods; and
wherein step b) is performed during second time periods; and
wherein the first and second time periods are non-overlapping.

25. Method (2300) of determining whether a balloon (133) of a balloon catheter (130) is positioned in a stomach (203) of a person (210), comprising the steps of:
i) performing (2301) the method of claim 17 as a first attempt to inflate the balloon to its nominal volume without the momentary pressure (pmom) becoming larger than the first threshold (p40);
ii) in case the balloon was deflated in step e) of the first attempt, performing (2302) the method of claim 18 or 19 or 20 as a second attempt to inflate the balloon to its nominal volume without the filtered pressure becoming larger than the second threshold (p30).

26. A computer program product comprising executable instructions, which, when being executed on a control unit (110) of a system (100) according to any of the claims 1 to 14, will perform a method according to any of the claims 15 to 25.
